(19) United States
(12) Patent Application Publication
(10) Pub. No.: US 2010/0222317 A1
(43) Pub. Date: Sep. 2, 2010

(54) Azetidine Derivatives as GlyT1 Inhibitors

(76) Inventors: Wesley Peter Blackaby, Epping (GB); Richard Thomas Lewis, Farmingham, MA (US); Elizabeth Mary Naylor, Needham, MA (US)

Correspondence Address: MERCK, P O BOX 2000, RAHWAY, NJ, 07065-0907, US

(21) Appl. No.: 12/085,340

(22) PCT Filed: Nov. 23, 2006
(86) PCT No.: PCT/GB2006/050411

§ 371 Date: May 14, 2009

(30) Foreign Application Priority Data
Nov. 25, 2005 (GB) 0523998.3

(51) Int. Cl. A61K 31/4427; C07D 213/56; A61K 31/4418; A61P 25/18; C07D 401/12; C07C 311/07; A61K 31/18; C07D 231/18; A61K 31/415; C07C 307/08; C07C 233/31; A61K 31/166; C07D 239/28; A61K 31/505

(52) U.S. Cl. 514/210.2; 546/337; 514/357; 546/268.1; 564/99; 514/605; 548/370.1; 514/407; 564/79; 564/186; 514/617; 544/335; 514/256

(57) ABSTRACT

The present invention relates to compounds of formula (I); and pharmaceutically acceptable salts thereof and individual enantiomers and diastereomers thereof, as GIyT1 inhibitors for treating neurological and psychiatric disorders.

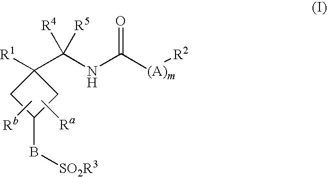

### BACKGROUND OF THE INVENTION

Schizophrenia is a debilitating psychiatric disorder characterized by a combination of negative (blunted affect, withdrawal, anhedonia) and positive (paranoia, hallucinations, delusions) symptoms as well as marked cognitive deficits. While the etiology of schizophrenia is currently unknown, the disease appears to be produced by a complex interaction of biological, environmental, and genetic factors. Over 40 years ago it was found that phencyclidine (PCP) induces a psychotic state in humans that is very similar to that observed in schizophrenic patients. The finding that the main mode of action of PCP is that of a non-competitive antagonist of the N-methyl-D-aspartate (NMDA) subtype of ionotropic glutamate receptor stimulated a series of studies that have led to the development of the NMDA receptor hypofunction model of schizophrenia (Jentsch J D and Roth R H, 1999 Neuropsychopharmacology, 20:201).

Fast glutamatergic transmission in the mammalian central nervous system is primarily mediated by the excitatory amino acid glutamate acting on ionotropic glutamate receptors (iGluRs). The iGluRs are comprised of three major subclasses, including the α-amino-3-hydroxy-5-methyl-4-isoxazolepropionic acid (AMPA), kainate, and NMDA receptor subtypes (Hollmann M and Heinemann S, 1994, Annu. Rev. Neurosci. 17:31). These three subclasses are multimeric ligand-gated cation channels which open in response to glutamate binding to induce a depolarizing excitatory post synaptic current. Molecular cloning has revealed that the NMDA receptor family is composed of two primary subunits, NR1 and NR2. In addition a novel inhibitory subunit which is developmentally regulated termed NR3 has been recently described. A high degree of molecular diversity exists within each set of subunits. To date, only one NR1 subunit gene has been cloned; however, alternative splicing of the NR1 gene can produce eight different subunits. In contrast, 4 genes have been cloned for the NR2 subunit (NR2A, NR2B, NR2C, and NR2D), some of which exhibit alternative splicing (Hollmann M and Heinemann S, 1994, Annu. Rev. Neurosci. 17:31). These multiple subunits form heteromeric glutamate-gated ion channels. While the precise subunit stoichiometry of the naturally occurring receptor remains unknown, both the NR1 and NR2 subunits are required for the expression of functionally active receptor-channel complexes in mammalian expression systems. Activation of the NMDA receptor requires the binding of both glutamate and glycine (Johnson J W and Ascher P, 1987, Nature 325:529). Interestingly, the binding sites for these two co-agonists exist on separate subunits as determined by site-directed mutagenesis studies (Laube B, Hirai H, Sturgess M, Betz H and Kuhse J, 1997, Neuron 18:493). On the NR2A and NR2B subunits, a binding pocket for glutamate is formed by interactions between the N-terminus of the receptor and the extracellular loops. Analogous experiments have placed the glycine binding site in a homologous region of the NR1 subunit (Kuryatov A, Laube B, Betz H and Kuhse J, 1994, Neuron 12:1291). Depending on the actual subunit composition, glutamate and glycine activate the NMDA receptor with EC50 values in the high nanomolar to low micromolar range. In addition, the pore of the NMDA receptor is impermeable to magnesium. Under normal resting conditions, extracellular magnesium can bind to a site within the pore and produce a magnesium block of the channel. This magnesium block imparts a strong voltage dependence to the channel which allows the NMDA receptor to act as a coincidence detector requiring the binding of glutamate, glycine, and the occurrence of postsynaptic depolarization before conducting current. Of particular interest is the finding that the psychotomimetic drugs MK-801, PCP, and ketamine all act as open channel blockers of the NMDA receptor-channel by binding to a site that overlaps with the magnesium binding site. It is apparent that the rich diversity of NMDA receptor subunits and regulatory sites provides for a complex assortment of physiologically and pharmacologically distinct heteromeric receptors making the NMDA receptor an ideal target for the design of novel therapeutic compounds.

The NMDA receptor plays a critical role in a variety of neurophysiological phenomena, including but not limited to synaptic plasticity, cognition, attention and memory (Bliss T and Collingridge W, 1993, Nature 361:31; Morris R G M et al., 1986, Nature 319:774). Psychotomimetic drugs constitute a wide class of drugs including psychomotor stimulants (cocaine, amphetamine), hallucinogens (LSD), and NMDA receptor antagonists (PCP, ketamine). Of these, only the NMDA receptor antagonists appear to elicit a robust induction of the positive, negative, and cognitive symptoms of schizophrenia. Controlled studies of ketamine-induced psychosis in human subjects, as well as observations of symptoms from patients abusing PCP as a recreational drug, have produced a convincing list of similarities between NMDA receptor antagonist-induced psychosis and schizophrenia (Jentsch J D and Roth R H, 1999 Neuropsychopharmacology, 20:201). NMDA-receptor antagonists faithfully mimic the symptoms of schizophrenia to the extent that it is difficult to differentiate the two in the clinic. In addition, NMDA receptor antagonists can exacerbate the symptoms in schizophrenics, and can trigger the re-emergence of symptoms in stable patients. Finally, the finding that NMDA receptor co-agonists such as glycine, D-cycloserine, and D-serine produce benefits in schizophrenic patients implicates NMDA receptor hypofunction in this disorder, and indicate that increasing NMDA receptor activation may provide a therapeutic benefit (Leiderman E et al., 1996, Biol. Psychiatry 39:213, Javitt D C et al., 1994, Am. J. Psychiatry 151:1234, Heresco-Levy U, 2000, Int. J. Neuropsychopharmacol. 3:243, Tsai G et al., 1998, Biol. Psychiatry 44:1081). A large number of studies in animal models lend support to the NMDA hypofunction hypothesis of schizophrenia. Recent generation of a mutant mouse expressing only 5% of normal levels of the NMDA NR1 subunit have shown that this decrease in functional NMDA receptors induces a state very similar to that observed in other animal models of schizophrenia (Mohn A R et al., 1999, Cell 98:427). Besides schizophrenia, dysfunction of glutamatergic pathways has been implicated in a number of disease states in the human central nervous system (CNS) including but not limited to cognitive deficits, dementia, Parkinson disease, Alzheimer disease and bipolar disorder.

NMDA receptor function can be modulated by altering the availability of the co-agonist glycine. This approach has the critical advantage of maintaining activity-dependent activation of the NMDA receptor because an increase in the synaptic concentration of glycine will not produce an activation of NMDA receptors in the absence of glutamate. Since synaptic glutamate levels are tightly maintained by high affinity transport mechanisms, an increased activation of the glycine site will only enhance the NMDA component of activated synapses. Clinical trials in which high doses of glycine were administered orally as an add-on to standard neuroleptic therapy showed an improvement of the symptoms of schizophrenia patients (Javitt et al. Int. J. Neuropsychopharmacol. (2001) 4: 385-391). One way to increase synaptic glycine levels without administering exogenous glycine is to inhibit its removal from the synapse. Evidence that this approach would be useful in treating schizophrenia comes from a double-blind placebo controlled study in which sarcosine was administered to patients suffering from schizophrenia, but who were poorly responsive to antipsychotic drugs. A beneficial effect was observed on positive, negative and cognitive symptoms, indicating that inhibition of glycine re-uptake is a reasonable approach to the treatment of schizophrenia.

Two specific glycine transporters, GlyT1 and GlyT2 have been identified and shown to belong to the Na⁺/Cl⁻ dependent family of neurotransmitter transporters which includes taurine, γ-aminobutyric acid (GABA), proline, monoamines and orphan transporters (Smith K E et al., 1992, Neuron 8:927; Borowsky B et al., 1993, Neuron 10:851; Liu Q R et al., 1993, J. Biol. Chem. 268:22802; Kim K M et al., 1994, Mol. Pharmacol. 45:608; Morrow J A et al., 1998, FEBS Lett. 439:334; Nelson N, 1998, J. Neurochem. 71:1785). GlyT1 and GlyT2 have been isolated from different species and shown to have only 50% identity at the amino acid level. They also have a different pattern of expression in mammalian central nervous system with GlyT2 being expressed in spinal cord, brainstem and cerebellum and GlyT1 present in these regions as well as forebrain areas such as cortex, hippocampus, septum and thalamus (Smith K E et al., 1992, Neuron 8:927; Borowsky B et al., 1993, Neuron 10:851; Liu Q R et al., 1993, J. Biol. Chem. 268:22802). At the cellular level, GlyT2 has been reported to be expressed by glycinergic nerve endings in rat spinal cord whereas GlyT1 appears to be preferentially expressed by glial cells (Zafra F et al., 1995, J. Neurosci. 15:3952). These expression studies have led to the conclusion that GlyT2 is predominantly responsible for glycine uptake at glycinergic synapses whereas GlyT1 is involved in monitoring glycine concentration in the vicinity of NMDA receptor expressing synapses. Recent functional studies in rat have shown that blockade of GlyT1 with the potent inhibitor (N-[3-(4′-fluorophenyl)-3-(4′-phenylphenoxy)propyl]sarcosine (NFPS) potentiates NMDA receptor activity and NMDA receptor-dependent long-term potentiation in rat (Bergeron R et al., 1998, PNAS USA 95:15730; Kinney G et al., 2003, J. Neurosci. 23:7586). Furthermore, NFPS has been reported to enhance pre-pulse inhibition in mice, a measure of sensory gating that is known to be deficient in schizophrenia patients (Kinney G et al., 2003, J. Neurosci. 23:7586). These physiological effects of GlyT1 in forebrain regions together with clinical reports showing the beneficial effects of GlyT1 inhibitor sarcosine in improving symptoms in schizophrenia patients (Tsai and Coyle WO99/52519) indicate that selective GlyT1 uptake inhibitors represent a new class of antipsychotic drugs.

Patent application WO03/063797 discloses that certain cycloalkyl derivatives are useful as inhibitors of potassium channel function for the treatment of disorders such as arrhythmia and IKur-associated disorders. A number of substituted N-[4-(N-substituted-N′-sulphenylureido)-1-phenyl-cyclohexylmethyl]-benzamides are described but there are no examples of the corresponding cyclobutyl compounds.

### SUMMARY OF THE INVENTION

The present invention is directed to compounds that inhibit the glycine transporter GlyT1 and which are useful in the treatment of neurological and psychiatric disorders associated with glutamatergic neurotransmission dysfunction and diseases in which the glycine transporter GlyT1 is involved.

### DETAILED DESCRIPTION OF THE INVENTION

The present invention is directed to compounds of the formula I:

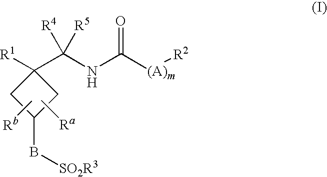

wherein R¹ is —(CH₂)ₙ—R^{1a}, wherein n is independently 0-6, and R^{1a} is selected from the group consisting of:
(1) C₁₋₆alkyl or C₁₋₆alkenyl, which is unsubstituted or substituted with 1-6 halogen, hydroxyl or —NR¹⁰R¹¹,
(2) phenyl substituted with R^{2a}, R^{2b} and R^{2c},
(3) heterocycle substituted with R^{2a}, R^{2b} and R^{2c},
(4) C₃₋₆cycloalkyl, which is unsubstituted or substituted with C₁₋₆alkyl, 1-6 halogen, hydroxy or —NR¹⁰R¹¹,
(5) —O—C₁₋₆alkyl, which is unsubstituted or substituted with 1-6 halogen, hydroxy or —NR¹⁰R¹¹,
(6) —CO₂R⁹,
   wherein R⁹ is independently selected from:
   (a) hydrogen,
   (b) —C₁₋₆alkyl, which is unsubstituted or substituted with 1-6 fluoro,
   (c) benzyl, and
   (d) phenyl,
(7) —NR¹⁰R¹¹,
   wherein R¹⁰ and R¹¹ are independently selected from:
   (a) hydrogen,
   (b) —C₁₋₆alkyl, which is unsubstituted or substituted with hydroxy, 1-6 fluoro or —NR¹²R¹³, where R¹² and R¹³ are independently selected from hydrogen and —C₁₋₆alkyl,
   (c) —C₃₋₆cycloalkyl, which is unsubstituted or substituted with hydroxy, 1-6 fluoro or —NR¹²R¹³,
   (d) benzyl,
   (e) phenyl, and
(8) —CONR¹⁰R¹¹;
   R² is selected from the group consisting of:
(1) phenyl, which is substituted with R^{2a}, R^{2b} and R^{2c},
(2) heterocycle, which is substituted with R^{2a}, R^{2b} and R^{2c},
(3) C₁₋₈alkyl, which is unsubstituted or substituted with 1-6 halogen, hydroxy, —NR¹⁰R¹¹, phenyl or heterocycle, where the phenyl or heterocycle is substituted with R^{2a}, R^{2b} and R^{2c},
(4) C₃₋₆cycloalkyl, which is unsubstituted or substituted with 1-6 halogen, hydroxy or —NR¹⁰R¹¹, and
(5) —C₁₋₆alkyl-(C₃₋₆cycloalkyl), which is unsubstituted or substituted with 1-6 halogen, hydroxy or —NR¹⁰R¹¹;
   R^{2a}, R^{2b} and R^{2c} are independently selected from the group consisting of:
(1) hydrogen,
(2) halogen,
(3) —C₁₋₆alkyl, which is unsubstituted or substituted with:
   (a) 1-6 halogen,
   (b) phenyl,
   (c) C₃₋₆cycloalkyl, or
   (d) —NR¹⁰R¹¹,
(4) —O—C₁₋₆alkyl, which is unsubstituted or substituted with 1-6 halogen,
(5) hydroxy,
(6) —SCF₃,
(7) —SCHF₂;
(8) —SCH₃,
(9) —CO₂R⁹,
(10) —CN,
(11) —SO₂R⁹,
(12) —SO₂—NR¹⁰R¹¹,
(13) —NR¹⁰R¹¹,
(14) —CONR¹⁰R¹¹, and
(15) —NO₂;
   R³ is selected from the group consisting of:
(1) C₁₋₆alkyl, which is unsubstituted or substituted with 1-6 halogen, hydroxyl, —NR¹⁰R¹¹, or heterocycle, which is substituted with R^{2a}, R^{2b} and R^{2c},
(2) C₃₋₆cycloalkyl, which is unsubstituted or substituted with 1-6 halogen, hydroxyl or —NR¹⁰R¹¹,
(3) —C₁₋₆alkyl-(C₃₋₆cycloalkyl), which is unsubstituted or substituted with 1-6 halogen, hydroxy or —NR¹⁰R¹¹,
(4) —NR¹⁰R¹¹, and
(5) heterocycle, which is substituted with R^{2a}, R^{2b} and R^{2c};
   R⁴ and R⁵ are each independently selected from the group consisting of:
(1) hydrogen, and
(2) C₁₋₆alkyl, which is unsubstituted or substituted with halogen or hydroxyl;
   A is selected from the group consisting of:
(1) —O—, and
(2) —NR¹⁰—;
   m is zero or one;
   B is selected from the group consisting of
(1) —CR⁶R⁷—, and
(2) —NR⁸
   wherein R⁶, R⁷ and R⁸ are each independently selected from hydrogen and C₁₋₆alkyl;
   R^{a} and R^{b} are each independently selected from hydrogen and C₁₋₄alkyl when B is NR⁸ and are each independently selected from hydrogen, fluorine, chlorine and C₁₋₄alkyl when B is CR⁶R⁷;
   and pharmaceutically acceptable salts thereof and individual enantiomers and diastereomers thereof.

In an embodiment, the present invention includes compounds wherein R¹ is selected from the group consisting of (CH₂)ₙR^{1a} wherein R^{1a} is C₃₋₆ cycloalkyl, which is unsubstituted or substituted with C₁₋₆alkyl, 1-6 halogen, hydroxy or —NR¹⁰R¹¹. In one embodiment, suitably n is 1 and R^{1a} is unsubstituted C₃₋₆ cycloalkyl, preferably cyclopropyl or cyclobutyl.

Further embodiments of the present invention include compounds wherein R¹ is heterocycle substituted with R^{2a}, R^{2b} and R^{2c}. The heterocycle is preferably an unsaturated heterocyclic moiety, for example a nitrogen containing unsaturated heterocycle such as pyridyl and R^{2a} and R^{2b} are hydrogen and R^{2c} is hydrogen or fluorine or a saturated heterocyclic moiety, for example a nitrogen containing saturated heterocycle such as piperidinyl, or pyrrolidinyl which is unsubstituted or substituted with R^{2a} and R^{2b} and R^{2c} is hydrogen wherein R^{2a} and R^{2b} are independently selected from the group consisting of C₁₋₆alkyl, 1-6 halogen, hydroxy, —O—C₁₋₆alkyl, or —NR¹⁰R¹¹, pyranyl, which is unsubstituted or substituted with C₁₋₆alkyl, 1-6 halogen, hydroxy, —O—C₁₋₆alkyl, or —NR¹⁰R¹¹, pyrrolyl, which is unsubstituted or substituted with C₁₋₆alkyl, 1-6 halogen, hydroxy, —O—C₁₋₆alkyl, or —NR¹⁰R¹¹, or azabicyclo[2.2.1]heptanyl, which is unsubstituted or substituted with C₁₋₆alkyl, 1-6 halogen, hydroxy, —O—C₁₋₆alkyl, or —NR¹⁰R¹¹.

An embodiment of the present invention includes compounds wherein R⁴ is C₁₋₃alkyl or hydrogen.

Also within this embodiment, the present invention includes compounds wherein R⁴ is hydrogen.

A further embodiment of the present invention includes compounds wherein R⁵ is C₁₋₃alkyl or hydrogen.

An embodiment of the present invention includes compounds wherein m is zero.

A further embodiment of the present invention includes compounds wherein R^{a} and R^{b} are each hydrogen.

Within this embodiment, the present invention includes compounds of the formula Ia:

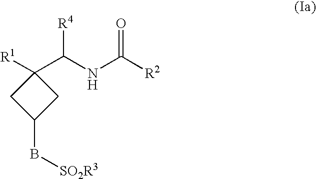

wherein B, R¹, R², R³ and R⁴ are as hereinbefore defined;
or a pharmaceutically acceptable salt thereof or an individual enantiomer or diastereomer thereof.

Further within this embodiment, the present invention includes compounds wherein R² is selected from the group consisting of:
(1) phenyl, which is substituted with R^{2a}, R^{2b} and R^{2c},
(2) heterocycle, such as pyridyl, pyrimidinyl or thienyl, which is substituted with R^{2a}, R^{2b} and R^{2c},
(3) C₁₋₈alkyl, which is unsubstituted or substituted with 1-6 halogen, phenyl or —NR¹⁰R¹¹, where the phenyl is substituted with R^{2a}, R^{2b} and R^{2c},
(4) C₃₋₆cycloalkyl, which is unsubstituted or substituted with 1-6 halogen, hydroxy or —NR¹⁰R¹¹, and
   R^{2a}, R^{2b} and R^{2c} are independently selected from the group consisting of:
(1) hydrogen,
(2) halogen,
(3) —C₁₋₆alkyl,
(4) —O—C₁₋₆alkyl,
(5) —CF₃,
(6) —OCF₃,
(7) —OCHF₂,
(8) —SCF₃,
(9) —SCHF₂, and
(10) —NH₂.

Also further within this embodiment, the present invention includes compounds wherein R² is phenyl, pyrimidyl or pyridyl substituted by R^{2a}, R^{2b} and R^{2c} as hereinbefore defined wherein at most only one of R^{2a}, R^{2b} and R^{2c} is hydrogen:

Within this embodiment the present invention includes compounds of the formula Ib:

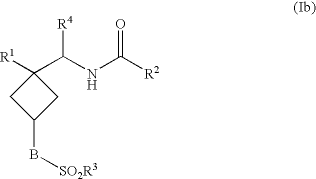

wherein R² is phenyl or unsaturated heterocycle substituted with R^{2a}, R^{2b} and R^{2c} and B, R¹, R³ and R⁴ are defined herein, B is CHR⁷ or NR⁸ and R^{2a}, R^{2b} and R^{2c} are selected from hydrogen, fluoro, chloro, bromo, CH₃, OCH₃, CF₃, OCF₃ and NH₂ and pharmaceutically acceptable salts thereof and individual enantiomers and diastereomers thereof. In one embodiment of this invention, B is CH₂. In a further aspect of this invention, B is NH or NCH₃. Preferably, when R² is an unsaturated heterocycle this is pyridyl or pyrimidyl.

An embodiment of the present invention includes compounds wherein R³ is a group R^{1a} and R^{3a} is a heterocycle as defined herein which is substituted with R^{2a}, R^{2b} and R^{2c}. Preferred heterocyclic groups R^{3a} include unsaturated heterocycles. Preferably the unsaturated heterocyle will be a six-membered ring containing one or more nitrogen atoms, for example pyridine, or a five-membered ring containing a sulphur atom or one to three nitrogen atoms, and preferably two or three nitrogen atoms, for example pyrazole.

Most suitably R^{3a} is a five-membered unsaturated heterocycle having one, two or three hetero atoms selected from one, two or three nitrogen atoms and additionally optionally an oxygen or sulphur atom that is linked to the sulphonyl group through one of the heterocycle's carbon atoms.

The unsaturated heterocycle may be unsubstituted or substituted by one or two halogen atoms or C₁₋₆ alkyl or C₁₋₆ haloalkyl groups. Preferably the unsaturated heterocycle is unsubstituted or substituted with one or two methyl or ethyl groups.

In another embodiment, R³ is a group, R^{3b} and R^{3b} is a C₁₋₄ alkyl group optionally substituted by halogen, for example one or two fluorine atoms, or by a C₃₋₆cycloalkyl group or R^{3b} is a group NR¹⁴R¹⁵ wherein R¹⁴ is hydrogen or a C₁₋₆ alkyl group and R¹⁵ is a C₁₋₆ alkyl group or R¹⁴ and R¹⁵ together with the nitrogen atom to which they are attached form a four to six membered heterocyclic ring.

In one embodiment, R^{3b} is a group NR¹⁴R¹⁵ wherein R¹⁴ is hydrogen or a C₁₋₆ alkyl group and R¹⁵ is a C₁₋₆ alkyl group or R¹⁴ and R¹⁵ together with the nitrogen atom to which they are attached form a four to six membered heterocyclic ring.

In one embodiment, B is a CH₂ or NH group.

A preferred group of compounds of the formula (I) is that of the formula Ic:

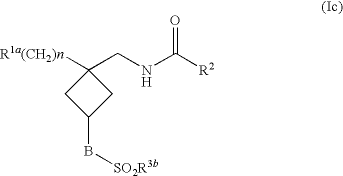

wherein R² is phenyl or unsaturated heterocycle substituted with R^{2a}, R^{2b} and R^{2c} and n, B, R^{1a} and R^{2a}, R^{2b} and R^{2a} are as hereinbefore defined and R^{3b} is C₁₋₄ alkyl group optionally substituted by a C₃₋₆cycloalkyl group.

n is preferably 0 or 1.

Preferred values of R^{1a} are as hereinbefore defined.

R^{2a}, R^{2b}, R^{2c} are preferably hydrogen, OCH₃, CH₃, CF₃ or halogen, suitably chlorine or fluorine. Preferably only one of R^{2a}, R^{2b}, R^{2c} is hydrogen.

A further preferred group of compounds of the formula (I) is that of the formula Id:

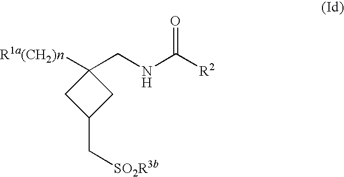

wherein R² is phenyl or unsaturated heterocycle substituted with R^{2a}, R^{2b} and R^{2c} and n, R^{1a} and R^{2a}, R^{2b}, R^{2c}, and R^{3b} are as hereinbefore defined.

R^{3b} is preferably a C₁₋₄ alkyl group optionally substituted by a cyclopropyl group, for example, a propyl or cyclopropylmethyl.

In one embodiment, when B is NH, R³ is not a group —NR¹⁰R¹¹ and R^{3b} is not a NR¹⁴R¹⁵ group, for example a mono C₁₋₄ alkylamino group.

Specific embodiments of the present invention include a compound which is selected from the group consisting of the subject compounds of the Examples herein and pharmaceutically acceptable salts thereof and individual enantiomers and diastereomers thereof.

The compounds of the present invention may contain one or more chiral centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, hexyl, heptyl and octyl. A group which is designated as being independently substituted with substituents may be independently substituted with multiple numbers of such substituents. The term “heterocycle” as used herein includes both unsaturated and saturated heterocyclic moieties, wherein the unsaturated heterocyclic moieties (i.e. “heteroaryl”) include benzoimidazolyl, benzimidazolonyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, and N-oxides thereof, and wherein the saturated heterocyclic moieties include azetidinyl, 1,4-dioxanyl, hexahydroazepinyl, piperazinyl, piperidinyl, pyridin-2-onyl, pyrrolidinyl, morpholinyl, tetrahydrofuranyl, thiomorpholinyl, and tetrahydrothienyl, and N-oxides thereof. The heterocycle may be bridged by a (1-3 alkylene group to form, for example, an azabicycloalkanyl group such as an azabicyclo[2.2.1]heptanyl group.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of inhibiting the glycine transporter GlyT1 activity in a patient such as a mammal in need of such inhibition comprising the administration of an effective amount of the compound. The present invention is directed to the use of the compounds disclosed herein as inhibitors of the glycine transporter GlyT1 activity. In addition to primates, especially humans, a variety of other mammals can be treated according to the method of the present invention.

The present invention is further directed to a method for the manufacture of a medicament for inhibiting glycine transporter GlyT1 activity in humans and animals comprising combining a compound of the present invention with a pharmaceutical carrier or diluent.

The subject treated in the present methods is generally a mammal, preferably a human being, male or female, in whom inhibition of glycine transporter GlyT1 activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with such disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy to retard the progression or reduce the risk of the noted conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibiting the glycine transporter activity, in particular GlyT1 activity, may be demonstrated by methodology known in the art. Human placental choriocarcinoma cells (JAR cells (ATCC No. HTB-144)) endogenously expressing GlyT1 were cultured in 96-well Cytostar scintillating microplates (Amersham Biosciences) in RPMI 1640 medium containing 10% fetal calf serum in the presence of penicillin (100 micrograms/milliliter) and streptomycin (100 micrograms/milliliter). Cells were grown at 37° C. in a humidified atmosphere of 5% CO2 for 40-48 hours before the assay. Culture medium was removed from the Cytostar plate, and JAR cells were incubated with 30 microliters of TB1A buffer (120 mM NaCl, 2 mM KCl, 1 mM CaCl₂, 1 mM MgCl₂, 10 mM HEPES, 5 mM L-alanine, pH 7.5 adjusted with Tris base) with or without the compounds of the present invention for 1 minute. Then 30 microliters of [¹⁴C]-glycine diluted with TB1A was added to each well to give a final concentration of 10 micromolar. After incubation at room temperature for 3 hours, the Cytostar scintillating microplates were sealed and counted on a Top Count scintillation counter (Packard). Non-specific uptake of [¹⁴C]-glycine was determined in the presence of 10 mM unlabeled glycine. [¹⁴C]taurine uptake experiments were performed according to the same protocol except that 10 mM unlabeled taurine was used to determine non-specific uptake. To determine potencies, a range of concentrations of the compounds of the present invention was added to the cells, followed by the fixed concentration of [¹⁴C]glycine. The concentration of the present compound that inhibited half of the specific uptake of [¹⁴C]glycine (IC₅₀ value) was determined from the assay data by non-linear curve fitting.

In particular, the compounds of the following examples had activity in inhibiting specific uptake of [¹⁴C]glycine in the aforementioned assay, generally with an IC₅₀ value of less than about 10 micromolar. Preferred compounds within the present invention had activity in inhibiting specific uptake of [¹⁴C]glycine in the aforementioned assay with an IC₅₀ value of less than about 1 micromolar. These compounds were selective for [¹⁴C]glycine uptake (by GlyT1 in the JAR cells) compared to [¹⁴C]taurine uptake (by the taurine transporter TauT in the JAR cells). Such a result is indicative of the intrinsic activity of the compounds in use as inhibitors of GlyT1 transporter activity.

The NMDA receptor is central to a wide range of CNS processes, and plays a role in a variety of disease states in humans or other species. The action of GlyT1 transporters affects the local concentration of glycine around NMDA receptors. Selective GlyT1 inhibitors slow the removal of glycine from the synapse, causing the level of synaptic glycine to rise. This in turn increases the occupancy of the glycine binding site on the NMDA receptor, which increases activation of the NMDA receptor following glutamate release from the presynaptic terminal. Because a certain amount of glycine is needed for the efficient functioning of NMDA receptors, any change to that local concentration can affect NMDA-mediated neurotransmission. Changes in NMDA-mediated neurotransmission have been implicated in certain neuropsychiatric disorders such as dementia, depression and psychoses, for example schizophrenia, and learning and memory disorders, for example attention deficit disorders and autism.

The compounds of the present invention have utility in treating a variety of neurological and psychiatric disorders associated with glutamatergic neurotransmission dysfunction, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; NMDA receptor-related disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Of the disorders above, the treatment of schizophrenia, bipolar disorder, depression including unipolar depression, seasonal depression and post-partum depression, premenstrual syndrome (PMS) and premenstrual dysphoric disorder (PDD), learning disorders, pervasive developmental disorder including autistic disorder, attention disorders including Attention-Deficit/Hyperactivity Disorder, autism, tic disorders including Tourette's disorder, anxiety disorders including phobia and post traumatic stress disorder, cognitive disorders associated with dementia, AIDS dementia, Alzheimer's, Parkinson's, Huntington's disease, spasticity, myoclonus, muscle spasm, tinnitus and hearing impairment and loss are of particular importance.

In a specific embodiment, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating anxiety disorders, comprising: administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another specific embodiment, the present invention provides a method for treating schizophrenia or psychosis comprising: administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In another specific embodiment, the present invention provides a method for treating substance-related disorders and addictive behaviors, comprising: administering to a patient in need thereof an effective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another specific embodiment, the present invention provides a method for treating pain, comprising: administering to a patient in need thereof an effective amount of a compound of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

In another specific embodiment, the present invention provides a method for treating obesity or eating disorders associated with excessive food intake and complications associated therewith, comprising: administering to a patient in need thereof an effective amount of a compound of the present invention. At present, obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-IV-TR. The skilled artisan will recognize that there are alternative nomenclatures, nosologies and classification systems for general medical conditions, and that these systems evolve with medical and scientific progress. Thus the term “obesity or eating disorders associated with excessive food intake” is intended to include like conditions and disorders that are described in other diagnostic sources.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents, including an inhibitor of glycine transporter GlyT1 activity.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention is preferred. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention.

The above combinations include combinations of a compound of the present invention not only with one other active compound, but also with two or more other active compounds Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexyl)hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexyl, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art.

In the treatment of conditions which require inhibition of glycine transporter GlyT1 activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Abbreviations used in the description of the chemistry and in the Examples that follow are:

| | |
|---|---|
| CH₂Cl₂ | dichloromethane |
| DIEA | diisopropylethylamine |
| PS-DIEA | polystyrene diisopropylethylamine |
| PS-DMAP | polystyrene 4-N,N-dimethylaminopyridine |
| DCC | polystyrene dicyclohexylcarbodiimide |
| Ra-Ni | Raney Nickel |
| HOBt | hydroxybenzotriazole |
| THF | tetrahydrofuran |
| TFA | trifluoroacteic acid |
| MeOH | methanol |
| LAH | lithium aluminium hydride |
| KHMDS | potassium bis(trimethylsilyl)amide |
| MsCl | methane sulphonyl chloride |
| PMBCl | p-methoxybenzyl chloride |
| CAN | ceric ammonium nitrate |
| Py | pyridine |
| TMSI | trimethylsilyl iodide |
| DIAD | diisopropyl azodicarboxylate |

Several methods for preparing the compounds of this invention are illustrated in the following Schemes and Examples. Starting materials and the requisite intermediates are in some cases commercially available, or can be prepared according to literature procedures or as illustrated herein.

The compounds of this invention may be prepared by employing methods well known to those skilled in the art for preparing analogous compounds, for example using the reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

The compounds of the formula (I) may be prepared by the acylation of the corresponding compound of the formula (II):

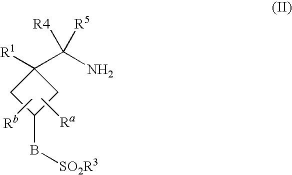

This acylation is conveniently carried out by the reaction of a compound of the formula (II) with a reactive derivative of a compound R²(A)mCOOH, for example an acid halide of the formula R²COhal, and preferably the appropriate acid chloride, in the presence of a weak base such as a trialkylamine, for example triethylamine, in a non polar solvent, for example a halogenated hydrocarbon such as dichloromethane, at a non-extreme temperature, for example −20 to 100° C. and conveniently 0 to 50° C.

The compounds of the formula (II) wherein B is NR⁸ may be prepared by reaction Scheme I as illustrated for compounds where R¹ is cyclopropylmethyl:

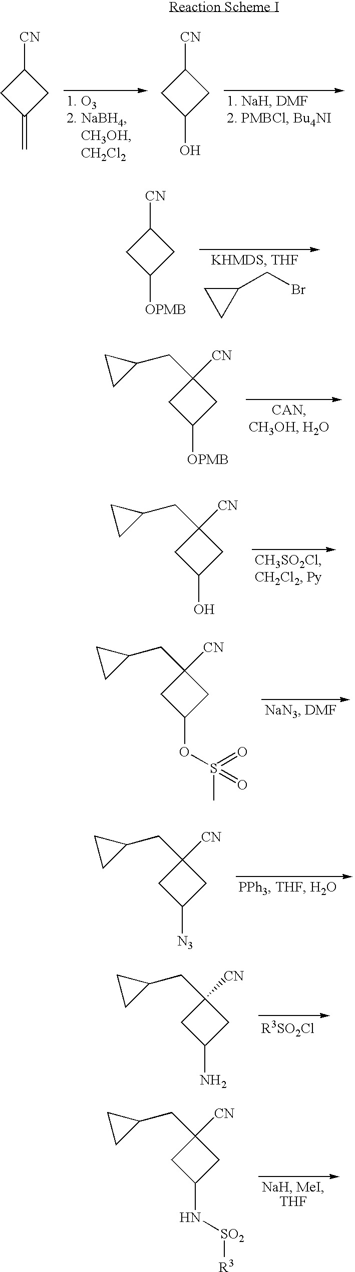

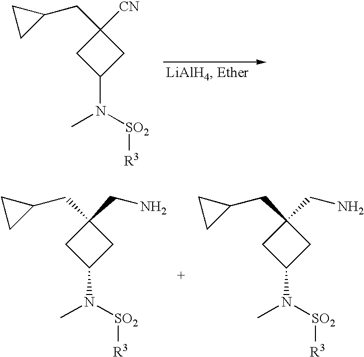

The compounds of the formula (II) where B is CR⁶R⁷ may be prepared by reaction schemes (II), (III) and (IV) again illustrated for where R¹ is cyclopropylmethyl:

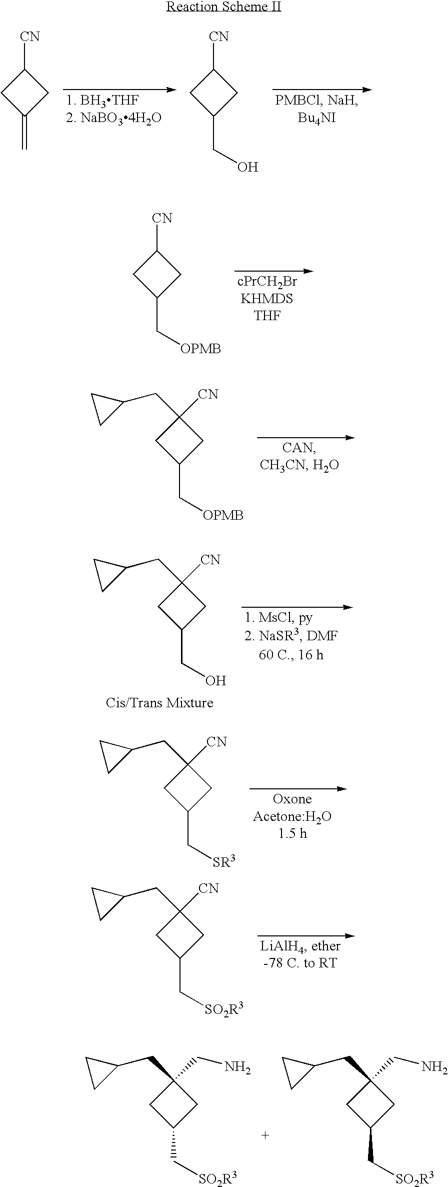

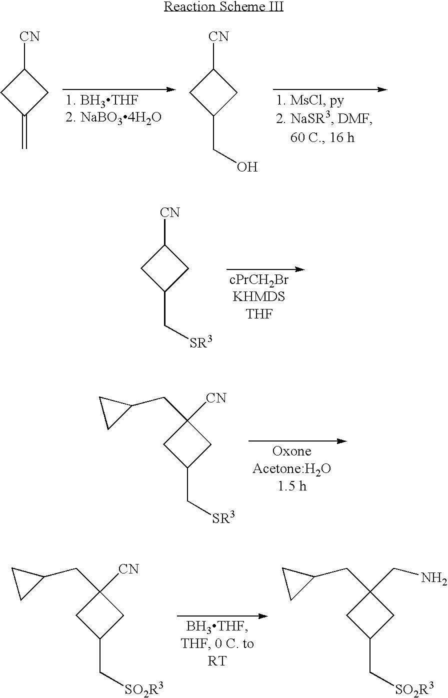

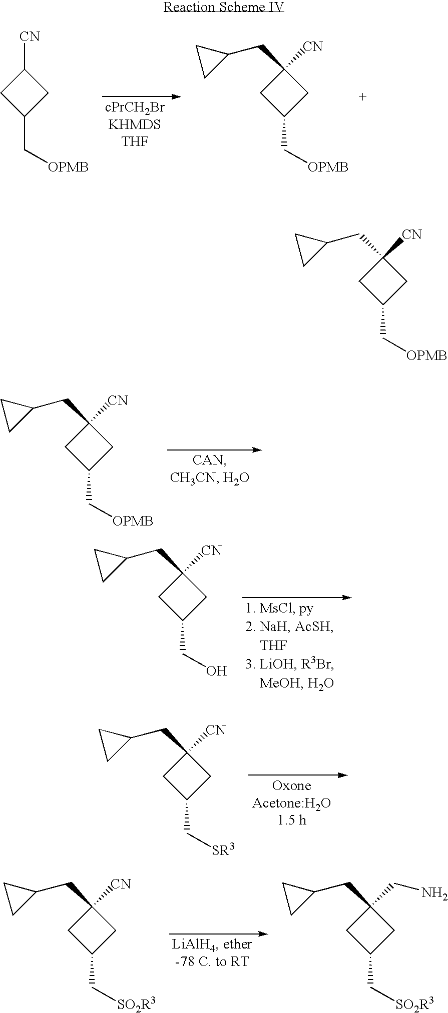

The compounds of the formula (I) may also be prepared by oxidation of the corresponding sulphanyl compound. This oxidation may conveniently be carried out by reaction with “Oxone” in a suitable solvent, for example a ketone such as acetone or TFA, at a non-extreme temperature, for example −20 to 150° C. and conveniently 20 to 100° C. When nitrogen containing heterocycles are present, it may be necessary to protect the ring nitrogen atom, for example with BOC, and then remove the protecting group after oxidation.

The sulphanyl compounds may be prepared by the method depicted in reaction Schemes V and VI wherein R¹ is illustrated as cyclopropyl and piperidine respectively

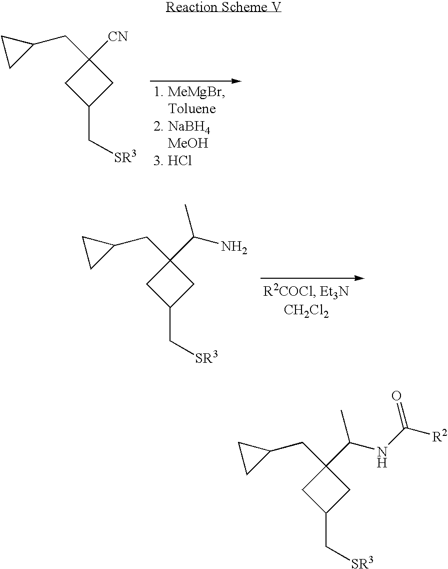

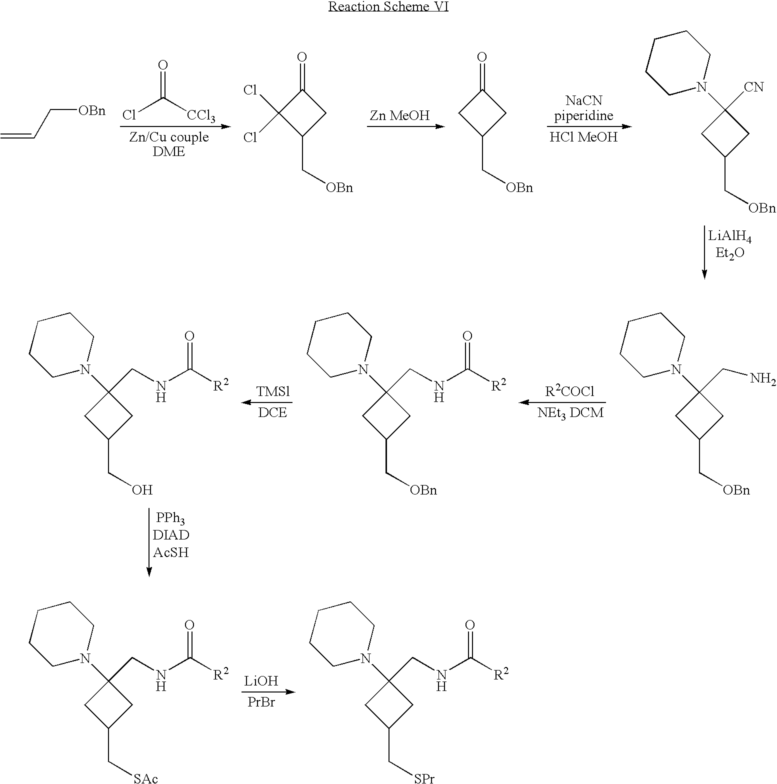

The compounds of the formula (I) wherein B is NH may also be prepared by acylation of the corresponding amino compound of the formula (III):

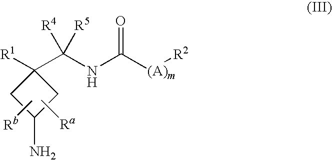

with an appropriately substituted sulphonyl compound. The sulphonyl compound will conveniently be the reactive derivative of a sulphonic acid, for example a sulphonyl halide such as a sulphonyl chloride.

This reaction may conveniently be carried out in the presence of a weak base, such as a trialkylamino, in a non-polar solvent, for example a halogenated hydrocarbon, such as methylene chloride.

The preparation of compounds of the formula (III) is illustrated by reaction Schemes (VII) and (VIII):

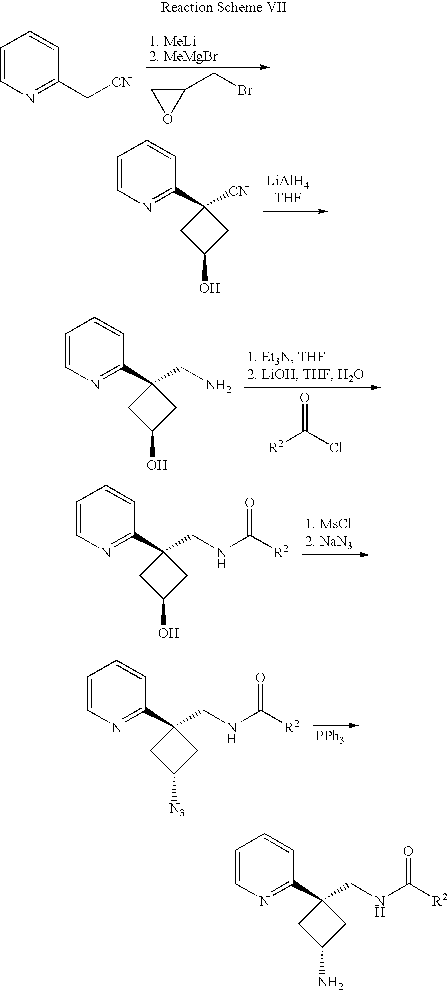

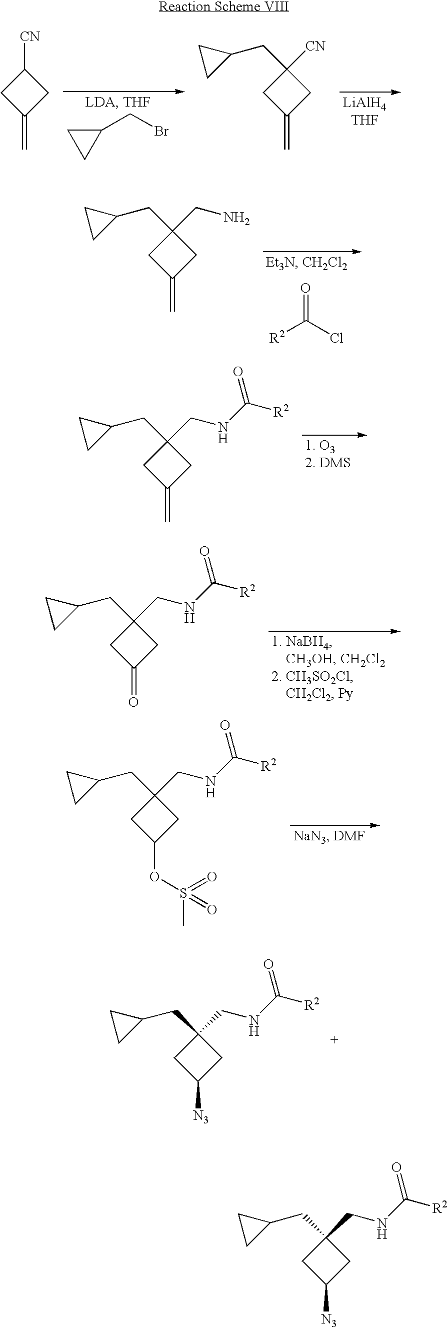

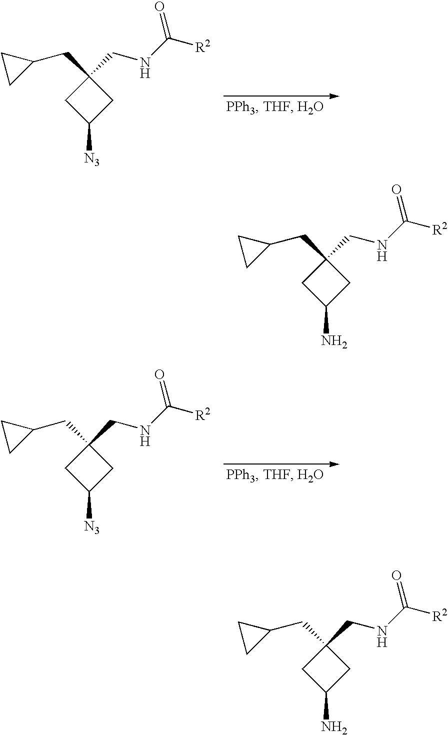

The following examples serve to illustrate the preparation of compounds of the present invention:

### Example 1

### Trans 2,4-Dichloro-N-[3-(propane-1-sulfonylamino)-1-pyridin-2-yl-cyclobutylmethyl]-benzamide (compound 1)

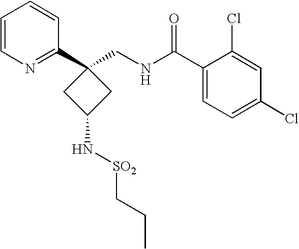

### Trans 3-Hydroxy-1-pyridin-2-yl-cyclobutanecarbonitrile

A solution of methyllithium (1.6M in diethyl ether) (25.6 mL, 41 mmol) was added dropwise to a stirred solution of pyridine acetonitrile (4.6 mL, 41 mmol) in THF (100 mL) at −78° C. After stirring for 1.5 hours, a solution of epibromohydrin (3.4 mL, 40 mmol) in THF (50 mL) was added dropwise over 0.5 hours. The reaction mixture was stirred at −78° C. for 1 hour then a solution of methyl magnesium bromide (3M in diethyl ether) (13.7 mL, 41 mmol) added dropwise. The reaction was allowed to warm slowly to ambient temperature over 12 hours then diluted with ethyl acetate (500 mL) and washed with brine (4×). The organic phase was dried (magnesium sulfate), filtered and evaporated to give a crude oil. Biotage chromatography (silica column) eluting with a 30-50% ethyl acetate-hexane afforded an oil (2.8 g, 39%) ¹H NMR (500 MHz, CD3OD): δ 8.60 (1H, d, J 4.6), 7.86-7.84 (1H, m), 7.58 (1H, d, J 7.9), 7.35 (1H, dd, J 4.9, 6.9), 4.63-4.57 (1H, m), 3.06-3.00 (2H, m), 2.76-2.70 (2H, m).

### Cis 3-Aminomethyl-3-pyridin-2-yl-cyclobutanol

A solution of lithium aluminium hydride (1.0M in THF) (1.42 mL, 1.42 mmol) was added to a solution of trans 3-hydroxy-1-pyridin-2-yl-cyclobutanecarbonitrile (225 mg, 1.29 mmol) in THF (5 mL) at ambient temperature. The solution was heated at reflux for 4 hours then cooled to ambient temperature. The reaction mixture was quenched by slow addition of ice then filtered through celite. The celite was washed with THF. The filtrate was evaporated to dryness and the residue azeotroped with toluene (3×) to leave an oil that was used in the next step without further purification MS (m/e)=179.

### Cis 2,4-Dichloro-N-(3-hydroxy-1-pyridin-2-yl-cyclobutylmethyl)-benzamide

Triethylamine (0.9 mL) followed by 2,4-dichlorobenzoyl chloride (0.9 mL, 6.4 mmol) was added to a solution of cis 3-aminomethyl-3-pyridin-2-yl-cyclobutanol (1.29 mmol) in THF (7 mL) at ambient temperature. The reaction mixture was stirred at ambient temperature for 48 hours then the volatile components evaporated. Ethyl acetate and brine were added and the organic phase separated. The organic phase was dried (magnesium sulfate), filtered and evaporated to leave a cream solid that was used without further purification.

The cream solid was dissolved in THF (4 mL) and water (2 mL). Lithium hydroxide (86 mg) was added to the solution. The solution was heated at 70° C. for 12 hours. After cooling to ambient temperature, lithium hydroxide (270 mg) was added and the reaction mixture heated at 70° C. for a further 3 hours. After cooling to ambient temperature, saturated ammonium chloride solution and ethyl acetate were added. The organic phase was separated and the aqueous phase re-extracted with ethyl acetate. The combined organic phase was washed with brine, dried (magnesium sulfate), filtered and evaporated to leave a crude oil. Biotage chromatography (silica column) eluting with a 1-6% methanol-methylene chloride afforded a glass (194 mg, 43% (3 steps)) ¹H NMR (500 MHz, CD3OD): δ 8.47 (1H, d, J 4.9), 7.80-7.76 (1H, m), 7.49 (1H, d, J 1.8), 7.37-7.30 (3H, m), 7.23 (1H, dd, J 5.0, 7.4), 4.51-4.45 (1H, m), 3.76 (2H, s), 2.84-2.80 (2H, m), 2.31-2.25 (2H, m); MS (m/e)=351.

### Trans N-(3-Azido-1-pyridin-2-yl-cyclobutylmethyl)-2,4-dichloro-benzamide

Methane sulfonyl chloride (0.058 mL, 0.75 mmol) was added to a solution of cis 2,4-dichloro-N-(3-hydroxy-1-pyridin-2-yl-cyclobutylmethyl)-benzamide (87 mg, 0.248 mmol) in pyridine (0.2 mL) and methylene chloride (1 mL) at ambient temperature. The solution was stirred for 4 hours then diluted with ethyl acetate. The solution was washed with brine (2×), dried (magnesium sulfate), filtered and evaporated to leave an oil (124 mg) that was used without further purification.

The oil was dissolved in DMF (1 mL). Sodium azide (33 mg, 0.51 mmol) was added and the reaction mixture heated at 120° C. with stirring for 5 hours. After cooling to ambient temperature, the mixture was diluted with ethyl acetate, washed with brine (3×), dried (magnesium sulfate), filtered and evaporated to leave an oil. The oil was purified by preparative thin layer chromatography eluting with 2% methanol-methylene chloride to give a cream solid (55 mg, 59% (2 steps)) ¹H NMR (500 MHz, CD3OD): δ 8.55 (1H, d, J 4.1), 7.83-7.79 (1H, m), 7.55 (1H, d, J 8.0), 7.50 (1H, d, J 1.8), 7.39-7.33 (2H, m), 7.27 (1H, dd, J 5.8, 7.4), 3.89-3.83 (1H, m), 3.73 (2H, s), 2.94-2.90 (2H, m), 2.51-2.47 (2H, m); MS (m/e)=376.

### Trans N-(3-Amino-1-pyridin-2-yl-cyclobutylmethyl)-2,4-dichloro-benzamide

Water (0.040 mL) followed by triphenylphosphine (111 mg, 0.423 mmol) was added to a solution of trans N-(3-azido-1-pyridin-2-yl-cyclobutylmethyl)-2,4-dichloro-benzamide (53 mg, 0.141 mmol) in THF (2 mL). The reaction mixture was stirred at ambient temperature for 12 hours then water was added. The reaction mixture was extracted with ethyl acetate (2×). The combined organic phase was washed with brine, dried (magnesium sulfate), filtered and evaporated to give an oil (45 mg, 91%) ¹H NMR (500 MHz, CD3OD): δ 8.54 (1H, d, J 3.8), 7.82-7.78 (1H, m), 7.57 (1H, t, J 8.3), 7.49 (1H, d, J 1.9), 7.36 (1H, dd, J 1.9, 8.2), 7.31-7.24 (2H, m), 3.74 (2H, s), 3.26-3.20 (1H, m), 2.93-2.87 (2H, m), 2.21-2.17 (2H, m); MS (m/e)=350.

### Trans 2,4-Dichloro-N-[3-(propane-1-sulfonylamino)-1-pyridin-2-yl-cyclobutylmethyl]-benzamide

Propyl sulfonyl chloride (0.016 mL, 0.14 mmol) was added dropwise to a stirred solution of trans N-(3-amino-1-pyridin-2-yl-cyclobutylmethyl)-2,4-dichloro-benzamide (24 mg, 0.070 mmol) and triethylamine (0.029 mL, 0.21 mmol) in methylene chloride (1 mL) cooled in an ice bath. The ice bath was removed and the reaction mixture stirred at ambient temperature for 5 hours. Methylene chloride and water were added and the organic phase separated. The aqueous phase was re-extracted with methylene chloride. The combined organic phase was washed with saturated sodium bicarbonate solution, brine, dried (magnesium sulfate), filtered and evaporated to leave an oil. The oil was purified by preparative thin layer chromatography eluting twice, first with 3% methanol-methylene chloride then with 5% methanol-methylene chloride to give a glass (8 mg, 25%) ¹H NMR (500 MHz, CD3OD): δ 8.56 (1H, d, J 4.0), 7.82 (1H, t, J 7.6), 7.60 (1H, d, J 7.9), 7.48 (1H, s), 7.36 (1H, d, J 8.2), 7.29 (2H, dd, J 8.4, 15.6), 3.75 (2H, s), 3.67 (1H, quin, J 8.2), 2.99-2.92 (4H, m), 2.39 (2H, t, J 10.5), 1.80-1.73 (2H, m), 1.04 (3H, t, J 7.4); analysis of COSY and NOESY spectra indicated trans regiochemistry; MS (m/e)=456.

### Example 2

### Trans N-[3-(Azetidine-1-sulfonylamino)-1-pyridin-2-yl-cyclobutylmethyl]-2,4-dichloro-benzamide (compound 2)

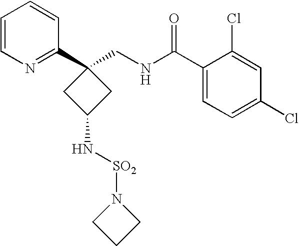

Diisopropylethylamine (0.043 mL, 0.249 mmol) followed by azetidine sulfamoyl chloride (19.4 mg, 0.125 mmol) were added to a solution of trans N-(3-amino-1-pyridin-2-yl-cyclobutylmethyl)-2,4-dichloro-benzamide (22 mg, 0.062 mmol) in methylene chloride (0.5 mL) cooled in an ice bath. The reaction mixture was allowed to warm to ambient temperature and stirred for 6 hours. Further diisopropylethylamine (0.043 mL, 0.249 mmol) and azetidine sulfamoyl chloride (19.4 mg, 0.125 mmol) were added and the reaction mixture stirred for 12 hours. The reaction mixture was directly purified by preparative thin layer chromatography eluting with methylene chloride-ethanol-ammonia (80:8:1) to give a glass (6.5 mg, 22%) ¹H NMR (500 MHz, CDCl3): δ 8.54 (1H, d, J 3.9), 7.73-7.65 (1H, m), 7.56 (1H, d, J 8.3), 7.48-7.44 (1H, m), 7.38 (1H, d, J 1.9), 7.30 (1H, m), 7.19 (1H, dd, J 5.7, 7.5), 7.13 (1H, m), 5.10 (1H, d, J 7.3), 3.97-3.83 (7H, m), 2.98-2.94 (2H, m), 2.40-2.36 (2H, m), 2.21-2.15 (2H, m); MS (m/e)=456.

### Example 3

### Cis 2,4-Dichloro-N-[1-cyclopropylmethyl-3-(propane-1-sulfonylamino)-cyclobutylmethyl]-benzamide (compound 3)

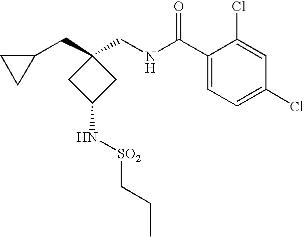

### 1-Cyclopropylmethyl-3-methylene-cyclobutanecarbonitrile

n-Butyllithium solution (2.5M in hexane) (19.3 mL, 0.048 mol) was added dropwise to diisopropylamine (6.77 mL, 0.048 mol) in THF (50 mL) cooled in an ice bath. After stirring for 0.5 hours, the solution was cooled to −78° C. and a solution of 3-methylenecyclobutanecarbonitrile (3.0 g, 0.032 mol) in THF (50 mL) was added dropwise. The orange solution was stirred at −78° C. for 1 hour then cyclopropylmethyl bromide (4.66 mL, 0.048 mol) added dropwise. The solution was allowed to warm to ambient temperature over 3 hours. The reaction mixture was quenched with water then extracted with methylene chloride (3×). The combined organic phase was washed with brine, dried (magnesium sulfate), filtered and evaporated to leave an oil. The oil was purified by Biotage Chromatography (silica column) eluting with 30% methylene chloride-hexane to give an impure oil (1.33 g, 28%) ¹H NMR (500 MHz, CDCl3): δ 4.94 (2H, d, J 2.2), 3.26 (2H, d, J 16.4), 2.82 (2H, d, J 16.4), 1.71 (2H, d, J 6.8), 0.87-0.81 (1H, m), 0.61-0.55 (2H, m), 0.26-0.19 (2H, m).

### C-(1-Cyclopropylmethyl-3-methylene-cyclobutyl)-methylamine

A solution of lithium aluminium hydride (1.0 M in diethyl ether) (16.3 mL, 16.3 mmol) was added dropwise to a solution of the 1-cyclopropylmethyl-3-methylene-cyclobutanecarbonitrile (1.20 g, 8.15 mmol) in diethyl ether (20 mL) cooled to −78° C. The reaction mixture was then allowed to warm slowly to ambient temperature over 3 hours then stirred at ambient temperature for 2 hours. The resultant white suspension was cooled in an ice bath and water (1 mL), followed by 15% sodium hydroxide solution (1 mL) then water (1 mL) added. After stirring for 0.25 hours at ambient temperature, the white granular precipitate was filtered. The white solid was washed with diethyl ether (3×). The filtrate was evaporated to leave a colourless oil (1.13 g, 92%) ¹H NMR (400 MHz, CDCl3): δ 4.80-4.78 (2H, m), 2.80 (2H, s), 2.47-2.35 (4H, m), 1.43 (2H, d, J 6.5), 0.70-0.56 (1H, m), 0.47-0.37 (2H, m), 0.09-0.00 (2H, m).

### 2,4-Dichloro-N-(1-cyclopropylmethyl-3-methylene-cyclobutylmethyl)-benzamide

Triethylamine (1.25 mL, 8.97 mmol) followed by 2,4-dichlorobenzoyl chloride (1.26 mL, 8.99 mmol) was added to a stirred solution of the C-(1-cyclopropylmethyl-3-methylene-cyclobutyl)-methylamine (1.13 g, 7.47 mmol) in methylene chloride (5 mL) and THF (5 mL) at ambient temperature. The white precipitate was stirred at ambient temperature for 1.5 hours. The reaction mixture was evaporated then partitioned between ethyl acetate and water. The organic phase was separated and washed with water, saturated sodium bicarbonate solution, brine, dried (magnesium sulfate), filtered and evaporated to leave an oil. The oil was purified by Biotage Chromatography (silica column) eluting with 5-10% ethyl acetate-hexane to give a white solid (1.29 g, 53%) ¹H NMR (400 MHz, CDCl3): δ 7.66 (1H, d, J 8.4), 7.43 (1H, d, J 2.0), 7.32 (1H, dd, J 2.0, 8.4), 6.27 (1H, s, N—H), 4.86-4.84 (2H, m), 3.68 (2H, d, J 5.8), 2.57 (4H, t, J 2.4), 1.51 (2H, d, J 6.9), 0.74-0.64 (1H, m), 0.51-0.47 (2H, m), 0.08 (2H, q, J 5.0).

### 2,4-Dichloro-N-(1-cyclopropylmethyl-3-oxo-cyclobutylmethyl)-benzamide

Ozone was bubbled into a solution of 2,4-dichloro-N-(1-cyclopropylmethyl-3-methylene-cyclobutylmethyl)-benzamide (1.29 g, 3.98 mmol) in methylene chloride (40 mL) and methanol (30 mL) at −78° C. until a blue coloration persisted. The solution was purged with oxygen then dimethyl sulfide (2.4 mL) was added with care. The reaction mixture was allowed to warm to ambient temperature over 12 hours. The reaction mixture was evaporated then the residue dissolved in ethyl acetate. The solution was washed with water twice, brine, dried (magnesium sulfate), filtered and evaporated to leave a white solid (1.22 g, 94%) ¹H NMR (400 MHz, CDCl3): δ 7.63 (1H, d, J 10.1), 7.44 (1H, d, J 1.9), 7.34-7.30 (1H, m), 6.40 (1H, s, N—H), 3.83 (2H, d, J 6.2), 3.10-2.91 (4H, m), 1.66 (2H, d, J 7.0), 0.78-0.68 (1H, m), 0.59-0.53 (2H, m), 0.16-0.12 (2H, m); MS (m/e)=326.

### 3-(Cyclopropylmethyl)-3-[((2,4-dichlorobenzoyl)-amino)methyl]cyclobutyl methanesulfonate

Sodium borohydride (77 mg, 2.04 mmol) was added to a solution of 2,4-dichloro-N-(1-cyclopropylmethyl-3-oxo-cyclobutylmethyl)-benzamide (600 mg, 1.84 mmol) in methylene chloride (15 mL) and methanol (15 mL) cooled in an ice bath. After stirring for 1 hour the reaction mixture was quenched with water then methylene chloride added. The organic phase was separated and the aqueous phase re-extracted with methylene chloride. The combined organic phase was washed with brine, dried (magnesium sulfate), filtered and evaporated to leave an oil (0.76 g) that was used without further purification.

The oil was dissolved in methylene chloride (10 mL) and pyridine (2 mL) was added. Methane sulfonyl chloride (0.427 mL, 5.52 mmol) was then added and the solution stirred for 12 hours. The reaction mixture was diluted with methylene chloride and the solution washed with water, 10% aqueous copper sulfate solution twice, water, brine, dried (magnesium sulfate), filtered and evaporated to leave an oil. The oil was purified by Biotage Chromatography (silica column) eluting with 30-40% ethyl acetate-hexane to give a colorless oil (0.70 g, 94%) ¹H (400 MHz, CDCl3): δ 7.66 (1H, d, J 8.3), 7.44 (1H, t, J 1.6), 7.34 (1H, dd, J 2.0, 8.4), 6.33 (1H, s, N—H), 5.14-5.04 (1H, m), 3.63 (2H, dd, J 3.6, 6.1), 2.98 (3H, d, J 5.7), 2.57-2.43 (2H, m), 2.34-2.20 (2H, m), 1.52-1.45 (2H, m), 0.70-0.66 (1H, m), 0.57-0.51 (2H, m), 0.13-0.07 (2H, m); MS (m/e)=406.

### Cis- and Trans-N-(3-Azido-1-cyclopropylmethyl-cyclobutylmethyl)-2,4-dichlorobenzamide

Sodium azide (112 mg, 1.72 mmol) was added to a stirred solution of 3-(cyclopropylmethyl)-3[((2,4-dichlorobenzoyl)amino)methyl]cyclobutyl methanesulfonate (350 mg, 0.861 mmol) in DMF (3.5 mL) under a nitrogen atmosphere at ambient temperature. The reaction mixture was heated at 100° C. for 5 hours. After cooling to ambient temperature, the DMF was evaporated and the residue partitioned between ethyl acetate and water. The organic phase was separated and washed with water, brine, dried (magnesium sulfate), filtered and evaporated to leave an oil. The oil was purified by Biotage Chromatography (silica column) eluting with 10-20% ethyl acetate-hexane to give two fractions as colourless oils. The higher running fraction, the cis isomer, (0.130 g) ¹H (400 MHz, CDCl3): δ 7.67 (1H, d, J 8.4), 7.43 (1H, d, J 2.0), 7.32 (1H, dd, J 2.0, 8.4), 6.29 (1H, s, N—H), 4.00-3.92 (1H, m), 3.59 (2H, d, J 6.1), 2.31-2.27 (2H, m), 2.06-2.02 (2H, m), 1.47 (2H, d, J 6.6), 0.72-0.64 (1H, m), 0.54-0.48 (2H, m), 0.12-0.06 (2H, m); MS (m/e)=353. The lower running fraction, the trans isomer (0.200 g) ¹H NMR (400 MHz, CDCl3): δ 7.67 (1H, d, J 8.3), 7.44 (1H, d, J 2.0), 7.33 (1H, dd, J 2.0, 8.3), 6.31 (1H, s, N—H), 4.03-3.95 (1H, m), 3.65 (2H, d, J 6.1), 2.36-2.32 (2H, m), 2.05-2.01 (2H, m), 1.46 (2H, d, J 6.8), 0.71-0.63 (1H, m), 0.54-0.50 (2H, m), 0.08 (2H, q, J 5.0); MS (m/e)=353. The cis and trans regiochemistry was assigned based upon the NOSEY spectra of compounds 3 and 4.

### Cis-N-(3-Amino-1-cyclopropylmethyl-cyclobutylmethyl)-2,4-dichlorobenzamide

Water (0.100 mL) followed by triphenyl phosphine (291 mg, 1.11 mmol) was added to a solution of the cis-N-(3-azido-1-cyclopropylmethyl-cyclobutylmethyl)-2,4-dichlorobenzamide (130 mg, 0.368 mmol) in THF (5 mL). The reaction mixture was stirred at ambient temperature for 12 hours. Water was added and the reaction mixture extracted with ethyl acetate twice. The combined organic phase was washed with brine, dried (magnesium sulfate), filtered and evaporated to leave an oil. The oil was purified by Biotage Chromatography (silica column) eluting with 5-7% 2M ammonia in methanol-methylene chloride to give a colourless oil (107 mg, 89%) ¹H NMR (400 MHz, CDCl3): δ 7.95 (1H, s, N—H), 7.60 (1H, d, J 8.3), 7.42 (1H, d, J 1.8), 7.30 (1H, dd, J 1.9, 8.3), 3.57-3.51 (3H, m), 2.32-2.23 (2H, m), 1.72-1.65 (2H, m), 1.46 (2H, d, J 6.4), 0.69-0.63 (1H, m), 0.51-0.47 (2H, m), 0.09 (2H, q, J 4.8). MS (m/e)=327.

### Cis 2,4-Dichloro-N-[1-cyclopropylmethyl-3-(propane-1-sulfonylamino)-cyclobutylmethyl]-benzamide (compound 3)

Propyl sulfonyl chloride (0.025 mL, 0.22 mmol) was added dropwise to a stirred solution of cis-N-(3-amino-1-cyclopropylmethyl-cyclobutylmethyl)-2,4-dichlorobenzamide (36 mg, 0.110 mmol) and triethylamine (0.046 mL, 0.33 mmol) in methylene chloride (1 mL). The solution was stirred for 48 hours then the volatile components evaporated to leave an oil. The oil was purified by preparative thin layer chromatography eluting with 3% methanol-methylene chloride to give a white solid (36 mg, 76%) ¹H NMR (500 MHz, CDCl3): δ 7.63 (1H, d, J 8.3), 7.43 (1H, d, J 1.9), 7.33 (1H, dd, J 2.0, 8.3), 6.38 (1H, s, N—H), 4.75 (1H, d, J 8.8), 3.99-3.91 (1H, m), 3.59 (2H, d, J 6.2), 2.93-2.89 (2H, m), 2.40-2.34 (2H, m), 1.98-1.94 (2H, m), 1.83-1.76 (2H, m), 1.48 (2H, d, J 6.6), 1.04 (3H, t, J 7.4), 0.70-0.63 (1H, m), 0.55-0.51 (2H, m), 0.11 (2H, q, J 4.9); MS (m/e)=433. The cis regiochemistry was assigned based upon the NOSEY spectra.

### Example 4

### Trans 2,4-Dichloro-N-[1-cyclopropylmethyl-3-(propane-1-sulfonylamino)-cyclobutylmethyl]-benzamide (compound 4)

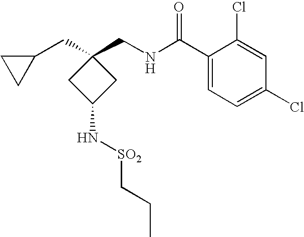

### Trans-N-(3-Amino-1-cyclopropylmethyl-cyclobutylmethyl)-2,4-dichlorobenzamide

Trans-N-(3-Amino-1-cyclopropylmethyl-cyclobutylmethyl)-2,4-dichlorobenzamide was synthesized from Trans-N-(3-Azido-1-cyclopropylmethyl-cyclobutylmethyl)-2,4-dichlorobenzamide using the procedure outlined above for Example 3.

¹H NMR (400 MHz, CDCl3): δ 7.66 (1H, d, J 8.4), 7.43 (1H, d, J 2.0), 7.32 (1H, dd, J 2.0, 8.3), 6.26 (1H, s, N—H), 3.63 (2H, d, J 5.8), 3.58-3.48 (1H, m), 2.34-2.28 (2H, m), 1.64-1.57 (2H, m), 1.42 (2H, d, J 6.7), 0.72-0.62 (1H, m), 0.50-0.46 (2H, m), 0.06 (2H, q, J 4.9); MS (m/e)=327.

### Trans 2,4-Dichloro-N-[1-cyclopropylmethyl-3-(propane-1-sulfonylamino)-cyclobutylmethyl]-benzamide (compound 4)

Trans 2,4-Dichloro-N-[1-cyclopropylmethyl-3-(propane-1-sulfonylamino)-cyclobutylmethyl]-benzamide (compound 4) was synthesized from trans-N-(3-amino-1-cyclopropylmethyl-cyclobutylmethyl)-2,4-dichlorobenzamide using the procedure outlined above for Example 3.

¹H NMR (400 MHz, CDCl3): δ 7.64 (1H, d, J 8.4), 7.42 (1H, d, J 2.0), 7.32 (1H, dd, J 2.0, 8.3), 6.30 (1H, s, N—H), 4.47 (1H, d, J 8.6), 4.04-3.94 (1H, m), 3.64 (2H, d, J 6.0), 2.95-2.91 (2H, m), 2.45-2.41 (2H, m), 1.90-1.76 (4H, m), 1.41 (2H, d, J 6.7), 1.04 (3H, t J 7.2), 0.68-0.60 (1H, m), 0.51-0.47 (2H, m), 0.083-0.026 (2H, m); MS (m/e)=433. The trans regiochemistry was assigned based upon the NOSEY spectra.

The following compounds were prepared by the methods outlined in examples 1-4 using the appropriate sulfonyl or sulfamoyl chloride and cis or trans N-(3-amino-1-cyclopropylmethyl-cyclobutylmethyl)-2,4-dichlorobensamide.

| Structure | MS data |
|---|---|
| 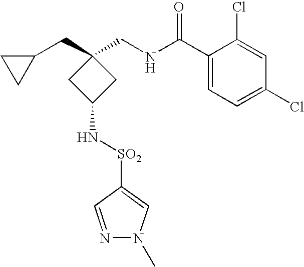 | 471 |
| 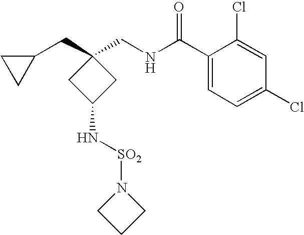 | 446 |
| 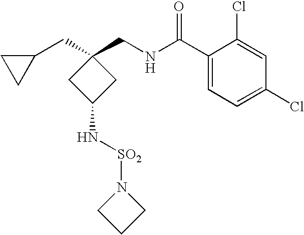 | 446 |
| 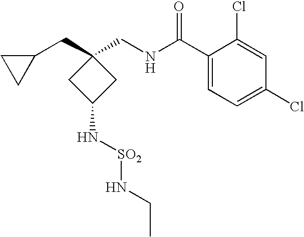 | 434 |
| 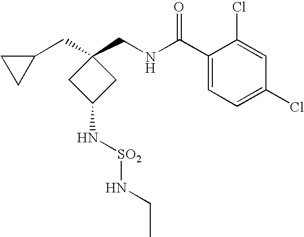 | 434 |
| 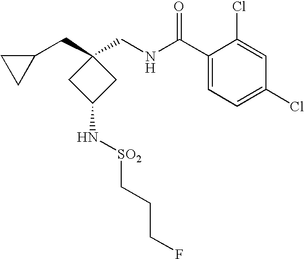 | 451 |
| 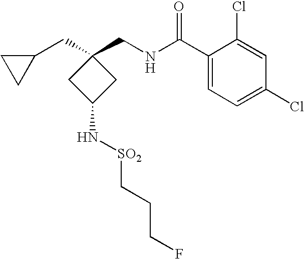 | 451 |

### Example 5

### Trans 2-Chloro-N-[1-cyclopropylmethyl-3-[methyl-(propane-1-sulfonyl)-amino]-cyclobutylmethyl]-4-trifluoromethyl-benzamide (compound 5)

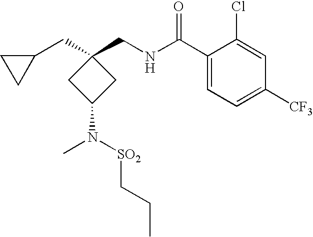

### 3-(4-Methoxy-benzyloxy)-cyclobutanecarbonitrile

Ozone was bubbled into a solution of 3-methylenecyclobutanecarbonitrile (2.00 g, 0.0215 mol) in methanol (150 mL) and methylene chloride (150 mL) at −78° C. until a blue colour persisted. Oxygen followed by nitrogen was bubbled through the solution then sodium borohydride (4.1 g, 0.108 mol) was added and the reaction mixture allowed to warm to ambient temperature over 12 hours. The solvent was evaporated and the residue partitioned between ethyl acetate and water. The organic phase was separated and the aqueous phase re-extracted with ethyl acetate twice. The combined organic phase was washed with brine, dried (magnesium sulfate), filtered, and the solvent evaporated to leave a pale yellow liquid (1.53 g, 73%) that was used in the next step without further purification.

A solution of 3-hydroxy-cyclobutanecarbonitrile in DMF (150 mL) was added dropwise to a suspension of sodium hydride (60% dispersion in oil) (1.26 g, 0.0315 mol) in DMF (50 mL) cooled in an ice bath. On completion of the addition, the ice bath was removed and the reaction mixture stirred at ambient temperature for 0.5 hours. A solution of p-methoxybenzyl chloride (4.25 mL, 0.0315 mol) and tetrabutylammonium iodide (2.91 g, 0.0079 mol) in DMF (50 mL) was added. The reaction mixture was stirred at ambient temperature for 12 hours then quenched with saturated ammonium chloride solution and extracted with diethyl ether (3×). The combined organic phase was washed with brine, dried (magnesium sulfate), filtered, and evaporated to leave a pale yellow liquid. The liquid was purified using the Horizon Chromatography system (silica column) eluting with 0-50% ethyl acetate-hexane to give a colourless liquid (2.95 g, 86%) ¹H NMR (400 MHz, CDCl3): δ 7.22 (2H, d, J 8.4), 6.88 (2H, d, J 8.4), 4.35 (2H, s), 4.00-3.91 (1H, m), 3.81 (3H, s), 2.67-2.55 (3H, m), 2.42-2.30 (2H, m).

### 1-Cyclopropylmethyl-3-(4-methoxy-benzyloxy)-cyclobutanecarbonitrile

Potassium bis(trimethylsilyl)amide solution (0.5M in toluene) (13.2 mL, 6.6 mmol) was added slowly dropwise to a stirred solution of 3-(4-methoxy-benzyloxy)-cyclobutanecarbonitrile (1.20 g, 5.52 mmol) and cyclopropylmethyl bromide (0.642 mL, 6.62 mmol) in THF (20 mL) at −78° C. The yellow solution was allowed to warm slowly to ambient temperature over 12 hours then quenched with saturated ammonium chloride solution and extracted with ethyl acetate (3×). The combined organic phase was washed with brine, dried (magnesium sulfate), filtered, and evaporated to leave a colourless oil. The oil was purified using the Horizon Chromatography System (silica column) eluting with 0-40% ethyl acetate-hexane to give a colourless oil (1.14 g, 76%) as a 1:1 mixture of cis and trans isomers ¹H NMR (500 MHz, CDCl3): δ 7.27-7.21 (2H, m), 6.88 (2H, d, J 7.7), 4.36 (1H, s), 4.35 (1H, s), 4.26 (0.5H, quin, J 7.3), 4.06 (0.5H, quin, J 7.3), 3.81 (3H, s), 2.79-2.75 (1H, m), 2.55-2.45 (2H, m), 2.10-2.04 (1H, m), 1.64 (1H, d, J 7.4), 1.59 (1H, d, J 7.0), 0.84-0.76 (1H, m), 0.57-0.50 (2H, m), 0.22-0.16 (2H, m).

### 1-Cyclopropylmethyl-3-hydroxy-cyclobutanecarbonitrile

Ceric ammonium nitrate (3.54 g, 6.46 mmol) was added to a solution of 1-cyclopropylmethyl-3-(4-methoxy-benzyloxy)-cyclobutanecarbonitrile (350 mg, 1.29 mmol) in acetonitrile (25 mL) and water (1 mL) with stirring and ice cooling. The reaction mixture was stirred at 0° C. for 0.5 hours then at ambient temperature for 1 hour. Saturated sodium bicarbonate solution was added and the reaction mixture extracted with methylene chloride (3×). The combined organic phase was washed with brine, dried (magnesium sulfate), filtered, and evaporated to leave an oil. The oil was purified using the Horizon Chromatography System (silica column) eluting with 0-40% ethyl acetate-hexane to give a colourless oil (145 mg, 74%) as a mixture of cis and trans isomers ¹H NMR (500 MHz, CDCl3): δ 4.62-4.56 (0.5H, m), 4.41-4.34 (0.5H, m), 2.89-2.85 (1H, m), 2.63-2.59 (1H, m), 2.52-2.48 (1H, m), 2.09-2.05 (1H, m), 1.97 (0.5H, s, O—H), 1.93 (0.5H, s, O—H), 1.67 (1H, d, J 7.2), 1.61 (1H, d, J 6.8), 0.86-0.78 (1H, m), 0.63-0.53 (2H, m), 0.24-0.18 (2H, m).

### 3-Amino-1-cyclopropylmethyl-cyclobutanecarbonitrile

Methane sulfonyl chloride (0.223 mL, 2.9 mmol) was added to a solution of 1-cyclopropylmethyl-3-hydroxy-cyclobutanecarbonitrile (145 mg, 0.96 mmol) in methylene chloride (5 mL) and pyridine (1 mL). The solution was stirred for 48 hours. The reaction mixture was diluted with methylene chloride and the solution washed with water, 10% aqueous copper sulfate solution twice, water, brine, dried (magnesium sulfate), filtered and evaporated to leave an oil (197 mg).

The oil was dissolved in DMF (3 mL) and sodium azide (112 mg, 1.72 mmol) added. The reaction mixture was heated at 50° C. for 6 hours then at 80° C. for 12 hours. After cooling to ambient temperature the reaction mixture was partitioned between ethyl acetate and water. The organic phase was separated and the aqueous phase re-extracted with ethyl acetate. The combined organic phase was washed with water, brine, dried (magnesium sulfate), filtered and evaporated to leave an oil (129 mg).

The oil as dissolved in THF (10 mL) and water (0.200 mL) followed by triphenyl phosphine (574 mg, 2.19 mmol) was added. The reaction mixture was stirred at ambient temperature for 48 hours. The THF was evaporated. The residue was purified by SCX cartridge (20 g) eluting with methanol (4×40 mL) then 2M ammonia in methanol (4×40 mL) to give a colourless oil (80 mg, 55%) as a 3:2 mixture of cis and trans isomers ¹H NMR (400 MHz, CDCl3): δ 3.80-3.72 (0.4H, m), 3.56-3.48 (0.6H, m), 2.85-2.81 (0.8H, m), 2.58-2.54 (1.2H, m), 2.26-2.22 (1.2H, m), 1.82-1.78 (0.8H, m), 1.65-1.60 (2H, m), 0.86-0.74 (1H, m), 0.59-0.51 (2H, m), 0.24-0.16 (2H, m); MS (m/e)=151.

### Cis and Trans Isomers of (3-Aminomethyl-3-cyclopropylmethyl-cyclobutyl)-methyl-amide propane-1-sulfonic acid

Propyl sulfonyl chloride (0.119 mL, 1.06 mmol) was added dropwise to a stirred solution of 3-amino-1-cyclopropylmethyl-cyclobutanecarbonitrile (80 mg, 0.53 mmol) and triethylamine (0.222 mL, 1.59 mmol) in methylene chloride (2.5 mL), cooled in an ice bath. The ice bath was removed and the reaction mixture stirred at ambient temperature for 12 hours. Methylene chloride and water were added and the organic phase separated. The aqueous phase was re-extracted with methylene chloride. The combined organic phase was washed with saturated sodium bicarbonate solution, brine, dried (magnesium sulfate), filtered and evaporated to leave an oil (120 mg).

The oil was dissolved in THF (5 mL) and sodium hydride (60% dispersion in oil) (47 mg, 1.18 mmol) was added in one portion. After stirring for 0.5 hours, methyl iodide (0.073 mL, 1.17 mmol) was added and the reaction mixture stirred at ambient temperature for 12 hours. Water was added and the reaction mixture extracted with methylene chloride twice. The combined organic phase was washed with brine, dried (magnesium sulfate), filtered and evaporated to leave an oil (114 mg).

The oil was dissolved in diethyl ether (2 mL) and cooled to −78° C. A solution of lithium aluminium hydride in diethyl ether (1.0 M) (0.84 mL, 0.84 mmol) was added dropwise then the reaction mixture allowed to warm to ambient temperature over 12 hours. Saturated sodium sulfate solution was then added and the reaction mixture stirred for 0.5 hours. Solid sodium sulfate was added and the mixture filtered and the filtrate evaporated to leave a pale yellow liquid. The liquid was purified using the Horizon Chromatography System (silica column) eluting with 0-10% methanol-methylene chloride to give the trans isomer (16 mg) ¹H NMR (400 MHz, CDCl3): δ 4.35-4.27 (1H, m), 2.86-2.82 (7H, m), 2.12-2.02 (4H, m), 1.85-1.75 (2H, m), 1.37 (2H, d, J 6.7), 1.04 (3H, t, J 7.5), 0.66-0.56 (1H, m), 0.50-0.46 (2H, m), 0.09-0.05 (2H, m) and the cis isomer (30 mg) ¹H NMR (400 MHz, CDCl3): δ 4.41-4.33 (1H, m), 2.85-2.81 (5H, m), 2.75 (2H, s), 2.10-2.00 (4H, m), 1.84-1.74 (2H, m), 1.46 (2H, d, J 6.6), 1.03 (3H, t, J 7.4), 0.66-0.58 (1H, m), 0.51-0.45 (2H, m), 0.12-0.07 (2H, m).

### Trans 2-Chloro-N-[1-cyclopropylmethyl-3-[methyl-(propane-1-sulfonyl)-amino]-cyclobutylmethyl]-4-trifluoromethyl-benzamide (compound 5)

2-Chloro-4-trifluoromethyl-benzoic acid (36.9 mg, 0.164 mmol) in thionyl chloride (1.5 mL) was heated at reflux for 1 hour. The thionyl chloride was evaporated and the residue azeotroped with toluene (3×) then the 2-chloro-4-trifluoromethyl-benzoyl chloride dried under vacuum. The benzoyl chloride was dissolved in methylene chloride (2 mL) and added to a stirred solution of the trans (3-aminomethyl-3-cyclopropylmethyl-cyclobutyl)-methyl-amide propane-1-sulfonic acid (15 mg, 0.0547 mmol) and triethylamine (0.038 mL, 0.273 mmol) in methylene chloride (1 mL). The reaction mixture was stirred at ambient temperature for 12 hours then the volatile components evaporated. The residue was partitioned between ethyl acetate and water. The organic phase was separated and the aqueous phase was re-extracted with ethyl acetate. The combined organic phase was washed with water, saturated sodium bicarbonate solution, brine, dried (magnesium sulfate), filtered and evaporated to leave an oil. The oil was purified using the Horizon Chromatography System (silica column) eluting with 0-80% ethyl acetate-hexane to give a colourless oil (23 mg) ¹H NMR (500 MHz, CDCl3): δ 7.80 (1H, d, J 8.0), 7.70 (1H, s), 7.61 (1H, d, J 8.0), 6.22 (1H, s), 4.40-4.33 (1H, m), 3.71 (2H, d, J 6.0), 2.88-2.82 (5H, m), 2.20 (4H, d, J 8.7), 1.85-1.77 (2H, m), 1.45 (2H, d, J 6.7), 1.05 (3H, t, J 7.4), 0.71-0.65 (1H, m), 0.56-0.50 (2H, m), 0.13-0.07 (2H, m); MS (m/e)=481. The trans regiochemistry was assigned based upon the NOSEY spectra.

### Example 6

### Cis 2-Chloro-N-[1-cyclopropylmethyl-3-[methyl-(propane-1-sulfonyl)-amino]-cyclobutylmethyl]-4-trifluoromethyl-benzamide (compound 6)

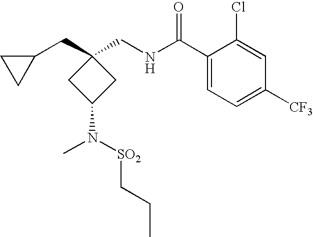

Cis 2-chloro-N-[1-cyclopropylmethyl-3-[methyl-(propane-1-sulfonyl)-amino]-cyclobutylmethyl]-4-trifluoromethyl-benzamide (compound 6) was synthesized from cis (3-aminomethyl-3-cyclopropylmethyl-cyclobutyl)-methyl-amide propane-1-sulfonic acid and 2-chloro-4-trifluoromethyl-benzoyl chloride using the procedure outlined above for Example 5.

¹H NMR (500 MHz, CDCl3): δ 7.77 (1H, d, J 8.0), 7.68 (1H, s), 7.59 (1H, d, J 8.0), 6.26 (1H, s), 4.37-4.30 (1H, m), 3.61 (2H, d, J 6.1), 2.85-2.79 (5H, m), 2.23-2.16 (2H, m), 2.15-2.11 (2H, m), 1.80-1.73 (2H, m), 1.52 (2H, d, J 6.3), 1.02 (3H, t, J 7.4), 0.72-0.65 (1H, m), 0.55-0.51 (2H, m), 0.11 (2H, q, J 5.0); MS (m/e)=481. The cis regiochemistry was assigned based upon the NOSEY spectra.

### Example 7

### Trans 2-Chloro-N-[1-cyclopropylmethyl-3-[(propane-1-sulfonylmethyl)-cyclobutylmethyl]-]-4-trifluoromethyl-benzamide (compound 7)

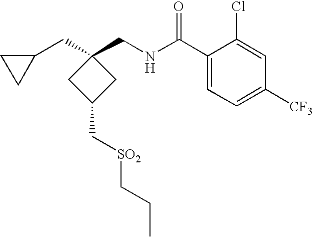

### 3-Hydroxymethyl-cyclobutanecarbonitrile

A solution of 9-BBN in THF (0.5M) (480 mL, 0.24 mol) was added over 1 hour to a stirred solution of 3-methylenecyclobutanecarbonitrile (18.6 g, 0.20 mol) in THF (800 mL) cooled in an ice bath. The reaction mixture was stirred for 0.25 hours then the ice bath removed and stirring continued for a further 4 hours. A suspension of sodium perborate (92.3 g) in water (1000 mL) was added slowly initially and then stirred for 12 hours. The reaction mixture was filtered and the solid washed with diethyl ether (3×). The filtrate was extracted with diethyl ether (3×). The combined organic phase was washed with brine, dried (magnesium sulfate), filtered, and evaporated to leave an oil. The oil was purified using the Horizon Chromatography System (silica column) eluting with 20-100% ethyl acetate-hexane to give a colourless oil (14.9 g, 67%) ¹H NMR (500 MHz, CDCl3): δ 3.67-3.61 (2H, m), 3.15-3.07 (0.3H, m), 3.06-2.96 (0.7H, m), 2.77-2.69 (0.3H, m), 2.62-2.51 (0.7H, m), 2.51-2.41 (2H, m), 2.60-2.36 (1H, m), 2.31-2.19 (2H, m).

### 3-(4-Methoxy-benzyloxymethyl)-cyclobutanecarbonitrile

A solution of 3-hydroxymethyl-cyclobutanecarbonitrile (1.64 g, 14.5 mmol) in DMF (50 mL) was added dropwise to a suspension of sodium hydride (60% dispersion in oil) (1.16 g, 0.0290 mol) in DMF (50 mL) cooled in an ice bath. On completion of the addition, the ice bath was removed and the reaction mixture stirred at ambient temperature for 0.5 hours. A solution of p-methoxybenzyl chloride (3.92 mL, 0.0290 mol) and tetrabutylammonium iodide (2.68 g, 0.0073 mol) in DMF (50 mL) was added. The reaction mixture was stirred at ambient temperature for 12 hours then quenched with saturated ammonium chloride solution and extracted with diethyl ether (3×). The combined organic phase was washed with brine, dried (magnesium sulfate), filtered, and evaporated to leave a pale yellow liquid. The liquid was purified by flash chromatography (silica column) eluting with 5-100% ethyl acetate-hexane to give a colourless liquid (2.95 g, 88%) ¹H NMR (400 MHz, CDCl3): δ 7.24 (2H, d, J 8.4), 6.90-6.88 (2H, m), 4.44 (2H, d, J 3.8), 3.81 (3H, s), 3.42 (0.8H, d, J 5.7), 3.40 (1.2H, d, J 6.1), 3.14-3.05 (0.4H, m), 3.02-2.92 (0.6H, m), 2.81-2.71 (0.4H, m), 2.65-2.54 (0.6H, m), 2.48-2.40 (2H, m), 2.29-2.17 (2H, m).

### 1-Cyclopropylmethyl-3-(4-methoxy-benzyloxymethyl)-cyclobutanecarbonitrile

Potassium bis(trimethylsilyl)amide solution (0.5M in toluene) (12.8 mL, 6.4 mmol) was added slowly dropwise to a stirred solution of 3-(methoxy-benzyloxymethyl)-cyclobutanecarbonitrile (1.23 g, 5.32 mmol) and cyclopropylmethyl bromide (0.62 mL, 6.39 mmol) in THF (20 mL) at −78° C. The yellow solution was allowed to warm slowly to ambient temperature over 12 hours then quenched with saturated ammonium chloride solution and extracted with ethyl acetate (3×). The combined organic phase was washed with brine, dried (magnesium sulfate), filtered, and evaporated to leave a colourless oil. The oil was purified using the Horizon Chromatography System (silica column) eluting with 5-50% ethyl acetate-hexane to give the trans isomer (0.16 g) ¹H NMR (500 MHz, CDCl3): δ 7.23 (2H, d, J 8.5), 6.88 (2H, d, J 8.5), 4.43 (2H, s), 3.81 (3H, s), 3.36 (2H, d, J 5.5), 2.90-2.82 (1H, m), 2.56-2.50 (2H, m), 2.02-1.96 (2H, m), 1.58 (2H, d, J 6.9), 0.81-0.73 (1H, m), 0.51 (2H, q, J 6.1), 0.15 (2H, q, J 5.1); a mixture of cis and trans isomers (0.37 g) ¹H NMR (500 MHz, CDCl3): δ 7.23 (2H, d, J 9.7), 6.88 (2H, dd, J 1.9, 8.6), 4.45 (1H, s), 4.43 (1H, s), 3.81 (3H, s), 3.48 (1H, d, J 6.5), 3.36 (1H, d, J 5.5), 2.90-2.82 (0.5H, m), 2.67-2.59 (0.5H, m), 2.56-2.50 (1H, m), 2.38-2.32 (1H, m), 2.28-2.22 (1H, m), 2.04-1.97 (1H, m), 1.68 (1H, d, J 6.8), 1.58 (1H, d, J 7.0), 0.86-0.72 (1H, m), 0.57-0.49 (2H, m), 0.22-0.14 (2H, m) and the cis isomer (0.46 g) ¹H NMR (500 MHz, CDCl3): δ 7.25 (2H, d, J 8.7), 6.88 (2H, d, J 8.5), 4.45 (2H, s), 3.81 (3H, s), 3.48 (2H, d, J 6.5), 2.66-2.51 (1H, m), 2.36-2.32 (2H, m), 2.27-2.21 (2H, m), 1.68 (2H, d, J 6.7), 0.85-0.77 (1H, m), 0.55 (2H, q, J 6.1), 0.21 (2H, q, J 5.1).

### 1-Cyclopropylmethyl-3-hydroxymethyl-cyclobutanecarbonitrile

Ceric ammonium nitrate (9.00 g, 16.4 mmol) was added to a solution of a mixture of cis and trans 1-cyclopropylmethyl-3-(4-methoxy-benzyloxymethyl)-cyclobutanecarbonitrile (936 mg, 3.28 mmol) in acetonitrile (50 mL) and water (5 mL) with stirring and ice cooling. The reaction mixture was stirred at 0° C. for 0.5 hours then at ambient temperature for 16 hours. Saturated sodium bicarbonate solution was added and the reaction mixture extracted with ethyl acetate (3×). The combined organic phase was washed with brine, dried (sodium sulfate), filtered, and evaporated to leave an oil. The oil was purified by flash chromatography (silica column) eluting with 20-100% ethyl acetate-hexane to give a colourless oil (479 mg, 88%) as a mixture of cis and trans isomers ¹H NMR (400 MHz, CDCl3): δ 3.70 (1.2H, d, J 6.4), 3.58 (0.8H, d, J 5.5), 2.91-2.73 (0.4H, m), 2.65-2.51 (1.4H, m), 2.39-2.35 (1.2H, m), 2.32-2.22 (1.2H, dd, J 2.3, 4.4), 2.04-1.98 (0.8H, m), 1.71 (1.2H, d, J 6.8), 1.61 (0.8H, d, J 6.9), 0.95-0.75 (1H, m), 0.62-0.50 (2H, m), 0.27-0.15 (2H, m).

### 1-Cyclopropylmethyl-3-propylsulfanylmethyl-cyclobutanecarbonitrile

Methane sulfonyl chloride (0.176 mL, 2.27 mmol) was added to a solution of 1-cyclopropylmethyl-3-hydroxymethyl-cyclobutanecarbonitrile (342 mg, 2.07 mmol) in pyridine (7 mL) cooled in an ice bath. The ice bath was removed and the solution stirred for 2 hours. The reaction mixture was evaporated to remove pyridine and the residue partitioned between ethyl acetate and water. The organic phase was separated and the aqueous phase re-extracted with ethyl acetate. The combined organic phase was washed with water, brine, dried (magnesium sulfate), filtered and evaporated to leave an oil.

The oil was dissolved in DMF (7 mL) and sodium propane thiolate (1.02 g, 10.4 mmol) added. The reaction mixture was heated at 60° C. for 16 hours. After cooling to ambient temperature, the reaction mixture was partitioned between diethyl ether and water. The organic phase was separated and the aqueous phase re-extracted with diethyl ether twice. The combined organic phase was washed with water (3×), brine, dried (magnesium sulfate), filtered and evaporated to leave an oil. The oil was purified using the Horizon Chromatography System (silica column) eluting with 10-100% methylene chloride-hexane to give a colourless liquid (0.381 g, 82%) as a mixture of cis and trans isomers ¹H NMR (500 MHz, CDCl3): δ 2.87-2.75 (0.4H, m), 2.67-2.60 (2H, m), 2.54-2.44 (3.4H, m), 2.35-2.25 (2.4H, m), 1.87-1.81 (0.8H, m), 1.66 (1.2H, d, J 6.8), 1.61-1.54 (2.8H, m), 0.96 (3H, t, J 7.3), 0.84-0.73 (1H, m), 0.56-0.49 (2H, m), 0.22-0.14 (2H, m).

### 1-Cyclopropylmethyl-3-(propane-1-sulfonylmethyl)-cyclobutanecarbonitrile

Oxone (3.15 g, 5.12 mmol) was added to a solution of 1-cyclopropylmethyl-3-propylsulfanylmethyl-cyclobutanecarbonitrile (381 mg, 1.71 mmol) in acetone (12 mL) and water (4 mL). The reaction mixture was heated at reflux for 1.5 hours. After cooling to ambient temperature, the reaction mixture was neutralized with 2N sodium carbonate solution and extracted with diethyl ether twice. The combined organic phase was washed with water, brine, dried (magnesium sulfate), filtered and evaporated to leave an oil (415 mg, 95%) ¹H NMR (400 MHz, CDCl3): δ 3.21-3.17 (1.6H, m), 3.08-3.00 (1.4H, m), 2.95-2.90 (2H, m), 2.85-2.79 (0.8H, m), 2.57-2.45 (2.4H, m), 2.10-2.04 (0.8H, m), 1.94-1.84 (2H, m), 1.74 (1.2H, d, J 6.8), 1.63 (0.8H, d, J 6.9), 1.11 (3H, t, J 7.4), 0.88-0.76 (1H, m), 0.61-0.53 (2H, m), 0.27-0.17 (2H, m).

### Cis and Trans 1-Cyclopropylmethyl-3-(propane-1-sulfonylmethyl-cyclobutyl)-methylamine

1-Cyclopropylmethyl-3-(propane-1-sulfonylmethyl)-cyclobutanecarbonitrile (415 mg, 1.62 mmol) was dissolved in diethyl ether (8 mL) and cooled to −78° C. A solution of lithium aluminium hydride in diethyl ether (1.0 M) (3.25 mL, 3.25 mmol) was added dropwise then the reaction mixture allowed to warm to ambient temperature over 12 hours. Saturated sodium sulfate solution was then added and the reaction mixture stirred for 0.5 hours. Solid sodium sulfate was added and the mixture filtered and the filtrate evaporated to leave a pale yellow liquid. The liquid was purified using the Horizon Chromatography System (silica column) eluting with 7-10% 2M ammonia in methanol-methylene chloride to give the trans isomer (109 mg) ¹H NMR (500 MHz, CDCl3): δ 3.07 (2H, d, J 7.1), 2.89-2.79 (5H, m), 2.15-2.11 (2H, m), 1.91-1.83 (2H, m), 1.77-1.69 (2H, m), 1.34 (2H, d, J 6.6), 1.08 (3H, t, J 7.4), 0.62-0.54 (1H, m), 0.49-0.43 (2H, m), 0.09-0.02 (2H, m) and the cis isomer (129 mg) ¹H NMR (500 MHz, CDCl3): δ 3.09 (2H, d, J 7.0), 2.90-2.84 (3H, m), 2.71 (2H, s), 2.16-2.10 (2H, m), 1.90-1.83 (2H, m), 1.76-1.70 (2H, m), 1.48 (2H, d, J 6.8), 1.08 (3H, t, J 7.4), 0.67-0.59 (1H, m), 0.51-0.45 (2H, m), 0.10-0.03 (2H, m). The cis and trans regiochemistry was assigned based upon the NOSEY spectra of compounds 7 and 8.

### Trans 2-Chloro-N-[1-cyclopropylmethyl-3-[(propane-1-sulfonylmethyl)-cyclobutylmethyl]-]-4-trifluoromethyl-benzamide (compound 7)

2-Chloro-4-trifluoromethyl-benzoic acid (118 mg, 0.525 mmol) in thionyl chloride (5 mL) was heated at reflux for 1 hour. The thionyl chloride was evaporated and the residue azeotroped with toluene (3×) then the 2-Chloro-4-trifluoromethyl-benzoyl chloride was dried under vacuum. The benzoyl chloride was dissolved in methylene chloride (2 mL) and added to a stirred solution of trans 1-cyclopropylmethyl-3-(propane-1-sulfonylmethyl-cyclobutyl)-methylamine (68 mg, 0.262 mmol) and triethylamine (0.11 mL, 0.79 mmol) in methylene chloride (6 mL). The reaction mixture was stirred at ambient temperature for 12 hours then the volatile components evaporated. The residue was partitioned between ethyl acetate and water. The organic phase was separated and the aqueous phase was re-extracted with ethyl acetate. The combined organic phase was washed with water, saturated sodium bicarbonate solution, brine, dried (magnesium sulfate), filtered and evaporated to leave an oil. The oil was purified using the Horizon Chromatography System (silica column) eluting with 10-90% ethyl acetate-hexane to give a oil (88 mg) ¹H NMR (500 MHz, DMSO): δ 8.54 (1H, t, J 5.7, N—H), 7.92 (1H, s), 7.76 (1H, d, J 7.8), 7.60 (1H, d, J 7.9), 3.48 (2H, d, J 5.9), 3.17 (2H, d, J 13.5), 2.97-2.92 (2H, m), 2.74-2.67 (1H, m), 2.15 (2H, t, J 10.6), 1.76-1.62 (4H, m), 1.35 (2H, d, J 6.5), 0.97 (3H, t, J 7.4), 0.71-0.63 (1H, m), 0.41-0.36 (2H, m), 0.06-0.01 (2H, m). The trans regiochemistry was assigned based upon the NOSEY spectra.

### Example 8

### Cis 2-Chloro-N-[1-cyclopropylmethyl-3-[(propane-1-sulfonylmethyl)-cyclobutylmethyl]-]-4-trifluoromethyl-benzamide (compound 8)

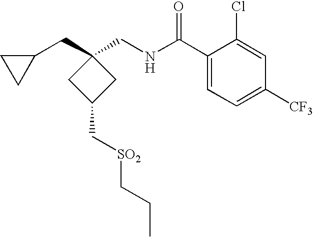

Cis 2-Chloro-N-[1-cyclopropylmethyl-3-[(propane-1-sulfonylmethyl)-cyclobutylmethyl]-]-4-trifluoromethyl-benzamide (compound 8) was synthesized from cis 1-cyclopropylmethyl-3-(propane-1-sulfonylmethyl-cyclobutyl)-methylamine and 2-chloro-4-trifluoromethyl-benzoyl chloride using the procedure outlined above for Example 7. ¹H NMR (500 MHz, DMSO): δ 8.52 (1H, t, J 5.9, N—H), 7.93 (1H, s), 7.77 (1H, d, J 7.7), 7.61 (1H, d, J 7.9), 3.34-3.31 (2H, m), 3.16 (2H, d, J 7.3), 2.95 (2H, t, J 7.8), 2.72-2.65 (1H, m), 2.01-1.95 (2H, m), 1.84 (2H, t, J 10.6), 1.69-1.62 (2H, m), 1.51-1.45 (2H, m), 0.96 (3H, t, J 7.4), 0.76-0.68 (1H, m), 0.44-0.38 (2H, m), 0.12-0.08 (2H, m). The cis regiochemistry was assigned based upon the NOSEY spectra.

### Example 9

### Cis and Trans N-[1-cyclopropylmethyl-3-[(propane-1-sulfonylmethyl)-cyclobutylmethyl]-]-2-methyl-6-trifluoromethyl-nicotinamide (compound 9)

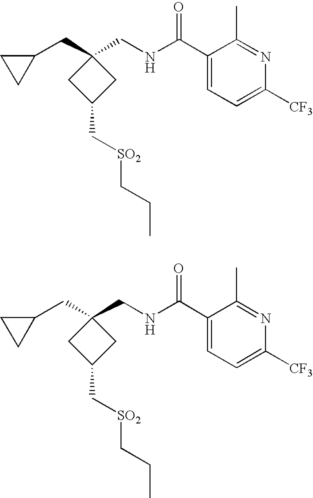

### 3-Propylsulfanylmethyl-cyclobutanecarbonitrile

Methane sulfonyl chloride (5.8 mL, 0.075 mol) was added to a solution of 3-hydroxymethyl-cyclobutanecarbonitrile (7.50 g, 0.0675 mol) in pyridine (100 mL) cooled in an ice bath. The ice bath was removed and the solution stirred for 2 hours. The reaction mixture was evaporated to remove pyridine and the residue partitioned between ethyl acetate and water. The organic phase was separated and the aqueous phase re-extracted with ethyl acetate. The combined organic phase was washed with water, brine, dried (magnesium sulfate), filtered and evaporated to leave an oil.

The oil was dissolved in DMF (100 mL) and sodium propane thiolate (33.1 g, 0.337 mol) added. The reaction mixture was heated at 60° C. for 16 hours. After cooling to ambient temperature, the reaction mixture was partitioned between diethyl ether and water. The organic phase was separated and the aqueous phase re-extracted with diethyl ether (4×). The combined organic phase was washed with water (5×), brine, dried (magnesium sulfate), filtered and evaporated to leave an oil. The oil was purified using the Horizon Chromatography System (silica column) eluting with 12-100% methylene chloride-hexane to give a colourless liquid (9.1 g, 80%) as a mixture of cis and trans isomers ¹H NMR (400 MHz, CDCl3): δ 3.10-2.74 (1H, m), 2.61-2.43 (7H, m), 2.20-2.06 (2H, m), 1.63-1.55 (2H, m), 0.97 (3H, t, J 7.3).

### 1-Cyclopropylmethyl-3-propylsulfanylmethyl-cyclobutanecarbonitrile

Potassium bis(trimethylsilyl)amide solution (0.5M in toluene) (129 mL, 0.0645 mol) was added slowly dropwise to a stirred solution of 3-propylsulfanylmethyl-cyclobutanecarbonitrile (9.1 g, 0.0538 mol) and cyclopropylmethyl bromide (6.35 mL, 0.0645 mol) in THF (200 mL) at −78° C. The yellow solution was allowed to warm slowly to ambient temperature over 12 hours then quenched with saturated ammonium chloride solution and extracted with ethyl acetate (3×). The combined organic phase was washed with brine, dried (magnesium sulfate), filtered, and evaporated to leave a colourless oil. The oil was purified using the Horizon Chromatography System (silica column) eluting with 7-50% ethyl acetate-hexane to give a colourless oil (8.8 g, 73%) as a mixture of cis and trans isomers ¹H NMR (400 MHz, CDCl3): δ 2.82-2.26 (8.5H, m), 1.88-1.84 (0.5H, m), 1.69-1.52 (4H, d, J 6.8), 0.98 (3H, t, J 7.3), 0.87-0.75 (1H, m), 0.59-0.51 (2H, m), 0.24-0.16 (2H, m).

### 1-Cyclopropylmethyl-3-(propane-1-sulfonylmethyl)-cyclobutanecarbonitrile

1-Cyclopropylmethyl-3-(propane-1-sulfonylmethyl)-cyclobutanecarbonitrile was synthesized from 1-cyclopropylmethyl-3-propylsulfanylmethyl-cyclobutanecarbonitrile using the procedure outlined above for Example 7.

### Cis and Trans C-1-[Cyclopropylmethyl-3-(propane-1-sulfonylmethyl)-cyclobutyl]-methylamine

Borane-THF complex (47 mL, 47 mmol) was added dropwise to a stirred solution of 1-cyclopropylmethyl-3-(propane-1-sulfonylmethyl)-cyclobutanecarbonitrile (2.0 g, 7.83 mmol) in THF (80 mL) cooled in an ice bath. After completion of the addition, the reaction mixture was allowed to warm to ambient temperature and stirred for 12 hours. The reaction mixture was added dropwise via a cannular to methanol cooled in an ice bath. On completion of the addition, concentrated hydrochloric acid (3.5 mL) was added and the mixture stirred at ambient temperature for 1 hour. The solvent was evaporated and the residue purified using an SCX cartridge eluting with methanol (3×), 2M ammonia in methanol (4×) to give a colourless oil (1.70 g, 84%) as a 2:1 mixture of cis and trans isomers ¹H NMR (400 MHz, CDCl3): δ 3.09-3.07 (2H, m), 2.89-2.69 (5H, m), 2.16-2.10 (2H, m), 1.91-1.83 (2H, m), 1.76-1.68 (2H, m), 1.48 (1.4H, d, J 6.5), 1.34 (0.6H, d, J 6.7), 1.08 (3H, t, J 7.4), 0.68-0.54 (1H, m), 0.50-0.44 (2H, m), 0.10-0.02 (2H, m).

### Cis and Trans N-[1-cyclopropylmethyl-3-[(propane-1-sulfonylmethyl)-cyclobutylmethyl]-]-2-methyl-6-trifluoromethyl-nicotinamide (compound 9)

A solution of 2-methyl-6-trifluoromethylnicotinoyl chloride (259 mg, 1.16 mmol) in methylene chloride (4 mL) was added to a solution of C-[1-cyclopropylmethyl-3-(propane-1-sulfonylmethyl)-cyclobutyl]-methylamine (250 mg, 0.964 mmol) and triethylamine (0.161 mL, 1.16 mmol) in methylene chloride (20 mL). The reaction mixture was stirred at ambient temperature for 1 hour. The volatile components were evaporated and the residue partitioned between ethyl acetate and water. The organic phase was separated and the aqueous phase re-extracted with ethyl acetate. The combined organic phase was washed with water, saturated sodium bicarbonate solution, brine, dried (magnesium sulfate), filtered and evaporated to leave an oil. The oil was purified using the Horizon Chromatography System (silica column) eluting with 50% ethyl acetate-hexane to give the trans isomer (15 mg) ¹H NMR (400 MHz, CDCl3): δ 7.80 (1H, d, J 7.9), 7.55 (1H, d, J 7.8), 5.96 (1H, s, N—H), 3.72 (2H, d, J 6.1), 3.07 (2H, d, J 7.4), 2.98-2.86 (3H, m), 2.72 (3H, s), 2.26-2.22 (2H, m), 1.91-1.81 (4H, m), 1.39 (2H, d, J 6.7), 1.09 (3H, t, J 7.4), 0.71-0.61 (1H, m), 0.54-0.48 (2H, m), 0.09-0.04 (2H, m); MS (m/e)=447 and a 3:2 cis:trans isomeric mixture (225 mg) and cis isomer (107 mg) ¹H NMR (400 MHz, CDCl3): δ 7.80 (1H, d, J 7.9), 7.57 (1H, d, J 7.5), 5.92 (1H, s, N—H), 3.59 (2H, d, J 6.1), 3.10 (2H, d, J 7.3), 2.98-2.86 (3H, m), 2.72 (3H, s), 2.26-2.18 (2H, m), 1.94-1.82 (4H, m), 1.55-1.53 (2H, m), 1.08 (3H, t, J 7.4), 0.72-0.66 (1H, m), 0.56-0.52 (2H, m), 0.14-0.08 (2H, m); MS (m/e)=447. The cis and trans regiochemistry was assigned based upon the NOSEY spectra.

The following compounds were prepared by the methods outlined in examples 7-9 using the appropriate acid chloride.

| Structure | MS data |
|---|---|
| 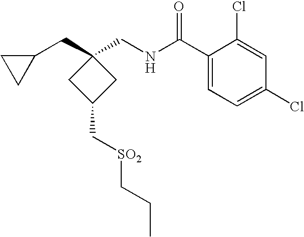 | 432 |
| 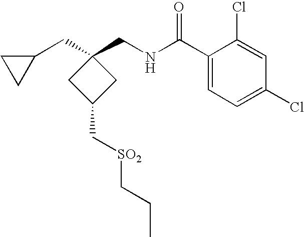 | 432 |
| 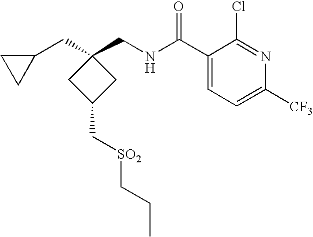 | 467 |
| 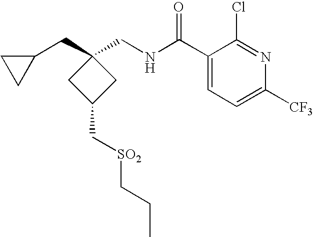 | 467 |
| 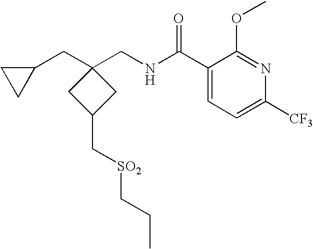 | 463 |
| 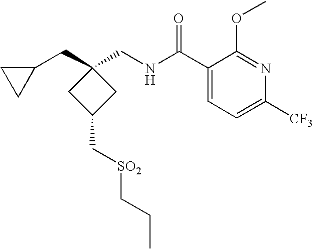 | 463 |
| 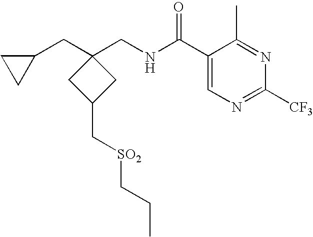 | 448 |
| 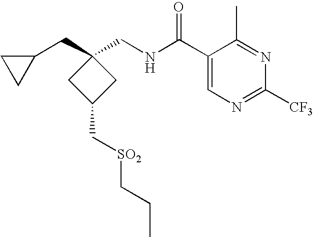 | 448 |
| 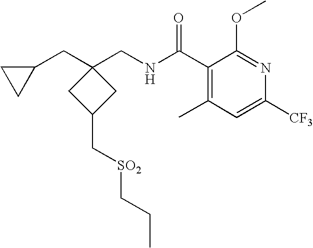 | 477 |
| 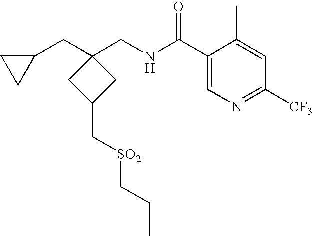 | 447 |
| 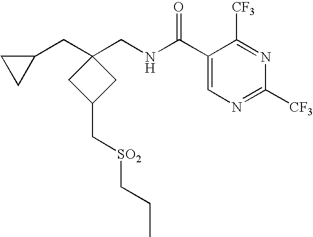 | 502 |
| 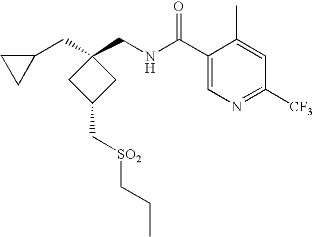 | 447 |
| 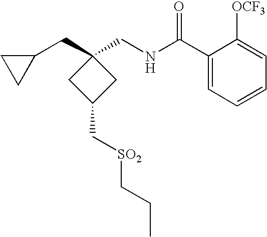 | 448 |
| 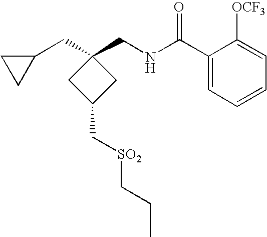 | 448 |
| 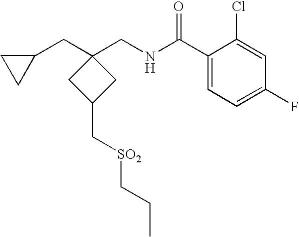 | 416 |
| 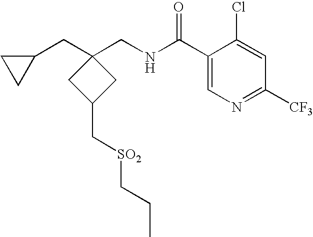 | 467 |
| 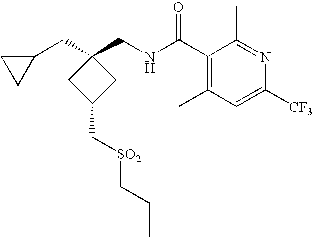 | 461 |
| 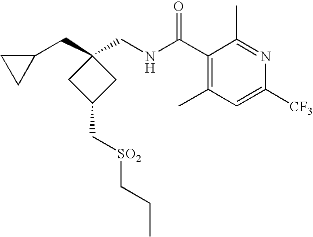 | 461 |
| 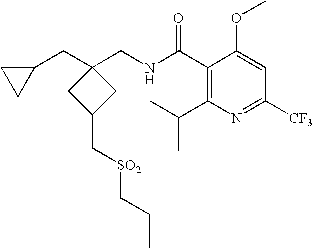 | 505 |
| 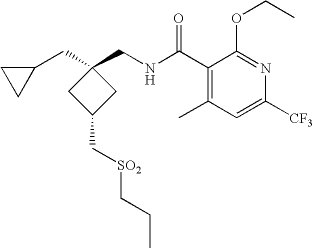 | 491 |
| 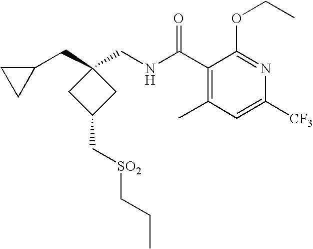 | 491 |
| 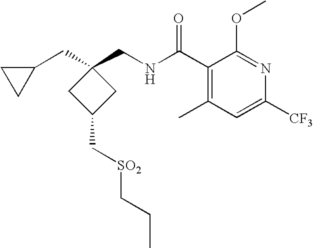 | 477 |
| 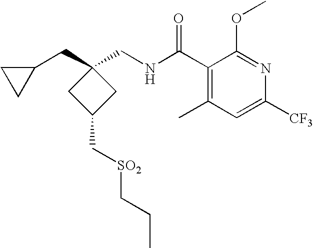 | 477 |
| 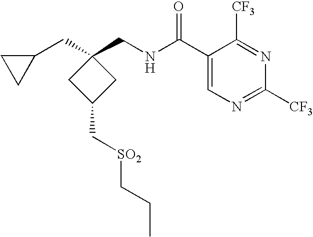 | 502 |
| 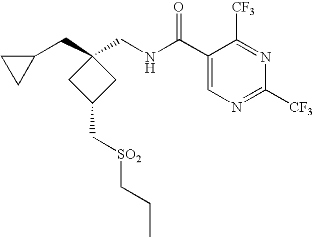 | 502 |

### Example 10

### Cis 2-Chloro-N-(3-cyclopropylmethanesulfonylmethyl-1-cyclopropylmethyl-cyclobutylmethyl)-4-trifluoromethyl-benzamide (compound 10)

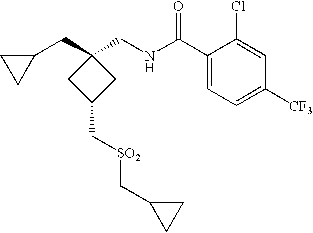

### Cis 1-Cyclopropylmethyl-3-hydroxymethyl-cyclobutanecarbonitrile

Ceric ammonium nitrate (3.68 g, 6.72 mmol) was added to a solution of cis 1-cyclopropylmethyl-3-(4-methoxy-benzyloxymethyl)-cyclobutanecarbonitrile (for synthesis see Example 7) (638 mg, 2.24 mmol) in acetonitrile (40 mL) and water (4 mL) with stirring and ice cooling. The reaction mixture was stirred at 0° C. for 0.5 hours then at ambient temperature for 1 hour. Saturated sodium bicarbonate solution was added and the reaction mixture extracted with methylene chloride (3×). The combined organic phase was washed with brine, dried (magnesium sulfate), filtered, and evaporated to leave an oil. The oil was purified by Horizon Chromatography (silica column) eluting with 5-65% ethyl acetate-hexane to give a colourless oil (346 mg, 94%) ¹H NMR (400 MHz, CDCl3): δ 3.74 (2H, d, J 6.4), 2.65-2.55 (1H, m), 2.42-2.38 (2H, m), 2.31-2.27 (2H, m), 1.73 (2H, d, J 6.8), 0.91-0.81 (1H, m), 0.61-0.57 (2H, m), 0.26 (2H, q, J 5.1).

### Cis 1-Cyclopropylmethyl-3-cyclopropylmethylsulfanylmethyl-cyclobutanecarbonittile

Methane sulfonyl chloride (0.077 mL, 0.99 mmol) was added to a solution of cis 1-cyclopropylmethyl-3-hydroxymethyl-cyclobutanecarbonitrile (150 mg, 0.91 mmol) in pyridine (3 mL) cooled in an ice bath. The ice bath was removed and the solution stirred for 2 hours. The reaction mixture was evaporated to remove pyridine and the residue partitioned between ethyl acetate and water. The organic phase was separated and the aqueous phase re-extracted with ethyl acetate. The combined organic phase was washed with 10% aqueous copper sulfate solution twice, brine twice, dried (magnesium sulfate), filtered and evaporated to leave the mesylate as an oil.

Thioacetic acid (0.325 mL, 4.55 mmol) was added to a suspension of sodium hydride (60% dispersion in oil) (200 mg, 5.0 mmol) in THF (5 mL) cooled in an ice bath. The ice bath was removed and the reaction mixture stirred at ambient temperature for 0.25 hours then re-cooled in an ice bath. A solution of the mesylate in THF (5 mL) was added and the reaction mixture stirred at ambient temperature for 12 hours then partitioned between diethyl ether and water. The organic phase was separated and the aqueous phase re-extracted with diethyl ether twice. The combined organic phase was washed with brine twice, dried (magnesium sulfate), filtered and evaporated to leave an orange oil (257 mg).

The oil was dissolved in methanol (2 mL) and water (0.2 mL). Cyclopropylmethyl bromide (0.354 mL, 3.6 mmol) followed by lithium hydroxide (88 mg, 3.6 mmol) were added and the reaction mixture stirred for 12 hours then poured into water and extracted with ethyl acetate twice. The combined organic phase was washed with brine, dried (magnesium sulfate), filtered and evaporated to leave an oil. The oil was purified by Horizon Chromatography (silica column) eluting with 5-45% ethyl acetate-hexane to give a colourless oil (95 mg, 45%) ¹H NMR (400 MHz, CDCl3): δ 2.76 (2H, d, J 7.6), 2.59-2.49 (1H, m), 2.44 (2H, d, J 7.4), 2.38-2.28 (4H, m), 1.68 (2H, d, J 6.8), 1.01-0.90 (1H, m), 0.89-0.77 (1H, m), 0.60-0.52 (4H, m), 0.24-0.16 (4H, m).

### Cis 3-Cyclopropylmethanesulfonylmethyl-1-cyclopropylmethyl-cyclobutanecarbonitrile

Cis 3-cyclopropylmethanesulfonylmethyl-1-cyclopropylmethyl-cyclobutanecarbonitrile was synthesized from cis 1-cyclopropylmethyl-3-cyclopropylmethylsulfanylmethyl-cyclobutanecarbonitrile, by oxidation with oxone, using the procedure outlined above for Example 7.

¹H NMR (400 MHz, CDCl3): δ 3.23 (2H, d, J 7.4), 3.10-3.02 (1H, m), 2.89 (2H, d, J 7.2), 2.57-2.47 (4H, m), 1.73 (2H, d, J 6.8), 1.22-1.12 (1H, m), 0.95-0.75 (3H, m), 0.60-0.54 (2H, m), 0.40 (2H, q, J 5.3), 0.24 (2H, q, J 5.1).

### Cis C-(3-Cyclopropylmethanesulfonylmethyl-1-cyclopropylmethyl-cyclobutyl)-methylamine

Cis C-(3-cyclopropylmethanesulfonylmethyl-1-cyclopropylmethyl-cyclobutyl)-methylamine was synthesized from cis 3-cyclopropylmethanesulfonylmethyl-1-cyclopropylmethyl-cyclobutanecarbonitrile, by reduction with lithium aluminium hydride, using the procedure outlined above for Example 7.

¹H NMR (400 MHz, CDCl3): δ 3.21 (2H, d, J 7.4), 2.94-2.84 (7H, m), 2.23-2.13 (2H, m), 1.84 (2H, t, J 10.9), 1.52 (2H, d, J 6.5), 1.19-1.13 (1H, m), 0.77-0.71 (2H, m), 0.69-0.55 (1H, m), 0.51-0.45 (2H, m), 0.39 (2H, q, J 5.3), 0.11 (2H, q, J 4.9); MS (m/e)=272.

### Cis 2-Chloro-N-(3-cyclopropylmethanesulfonylmethyl-1-cyclopropylmethyl-cyclobutylmethyl)-4-trifluoromethyl-benzamide (compound 10)

Cis 2-Chloro-N-(3-cyclopropylmethanesulfonylmethyl-1-cyclopropylmethyl-cyclobutylmethyl)-4-trifluoromethyl-benzamide (compound 10) was synthesized from cis C-(3-cyclopropylmethanesulfonyl methyl-1-cyclopropylmethyl-cyclobutyl)-methylamine and 2-chloro-4-trifluoromethyl-benzoyl chloride using the procedure outlined above for Example 7.

¹H NMR (500 MHz, CDCl3): δ 7.76 (1H, d, J 7.8), 7.69 (1H, s), 7.60 (1H, d, J 8.0), 6.27 (1H, s, N—H), 3.59 (2H, d, J 6.1), 3.16 (2H, d, J 7.3), 2.98-2.90 (1H, m), 2.84 (2H, d, J 7.1), 2.29-2.21 (2H, m), 1.94-1.84 (2H, m), 1.56 (2H, d, J 6.5), 1.21-1.11 (1H, m), 0.78-0.66 (3H, m), 0.54-0.47 (2H, m), 0.41-0.37 (2H, m), 0.12 (2H, q, J 4.9); MS (m/e)=478. The cis regiochemistry was assigned based upon the NOSEY spectra.

### Example 11

### Trans 2-Chloro-N-(3-cyclopropylmethanesulfonylmethyl-1-cyclopropylmethyl-cyclobutylmethyl)-4-trifluoromethyl-benzamide (compound 11)

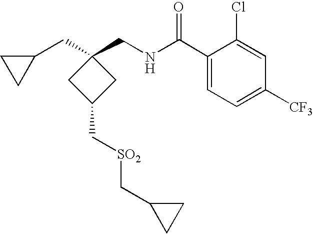

Trans 2-Chloro-N-(3-cyclopropylmethanesulfonylmethyl-1-cyclopropylmethyl-cyclobutylmethyl)-4-trifluoromethyl-benzamide (compound 11) was synthesized from trans 1-cyclopropylmethyl-3-(4-methoxy-benzyloxymethyl)-cyclobutanecarbonitrile (for synthesis see Example 7) using the procedure outlined above for Example 10.

¹H NMR (500 MHz, CDCl3): δ 7.79 (1H, d, J 8.0), 7.69 (1H, s), 7.60 (1H, d, J 8.0), 6.22 (1H, s, N—H), 3.75 (2H, d, J 5.9), 3.14 (2H, d, J 7.4), 2.99-2.92 (1H, m), 2.86 (2H, d, J 7.1), 2.27 (2H, t, J 10.7), 1.91-1.84 (2H, m), 1.42 (2H, d, J 6.6), 1.20-1.12 (1H, m), 0.76 (2H, q, J 6.3), 0.71-0.61 (1H, m), 0.54-0.48 (2H, m), 0.40 (2H, q, J 5.2), 0.07 (2H, q, J 4.9). The trans regiochemistry was assigned based upon the NOSEY spectra.

### Example 12

### Trans N-(3-cyclopropylmethanesulfonylmethyl-1-cyclopropylmethyl-cyclobutylmethyl)-2-methyl-6-trifluoromethyl-nicotinamide (compound 12)

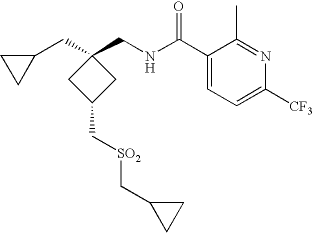

Trans N-(3-cyclopropylmethanesulfonylmethyl-1-cyclopropylmethyl-cyclobutylmethyl)-2-methyl-6-trifluoromethyl-nicotinamide (compound 12) was synthesized from trans 1-cyclopropylmethyl-3-(4-methoxy-benzyloxymethyl)-cyclobutanecarbonitrile (for synthesis see Example 7) using the procedure outlined above for Example 10.

¹H NMR (500 MHz, CDCl3): δ 7.81 (1H, d, J 7.8), 7.56 (1H, d, J 7.8), 5.92 (1H, s, N—H), 3.73 (2H, d, J 6.0), 3.15 (2H, d, J 7.8), 3.01-2.91 (1H, m), 2.85 (2H, d, J 7.3), 2.72 (3H, s), 2.27-2.23 (2H, m), 1.94-1.86 (2H, m), 1.40 (2H, d, J 6.7), 1.20-1.12 (1H, m), 0.77 (2H, q, J 6.4), 0.70-0.63 (1H, m), 0.54-0.48 (2H, m), 0.40 (2H, q, J 5.2), 0.07 (2H, q, J 4.9); MS (m/e)=459. The trans regiochemistry was assigned based upon the NOSEY spectra of compound 11.

### Example 13

### N-{1-[1-Cyclopropylmethyl-3-(propane-1-sulfonylmethyl)-cyclobutyl]-ethyl}-2-methoxy-4-methyl-6-trifluoromethyl-nicotinamide (compound 13)

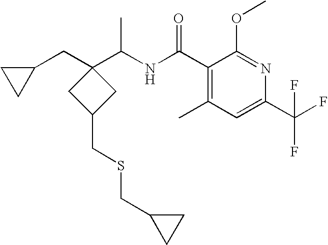

### 1-(1-Cyclopropylmethyl-3-propylsulfanylmethyl-cyclobutyl)-ethylamine

1-Cyclopropylmethyl-3-propylsulfanylmethyl-cyclobutanecarbonitrile (for synthesis see Example 9) (5.15 g, 23.05 mmol) was dissolved in toluene (30 mL) and methyl magnesium bromide solution (1.4 M in toluene/THF) (24.7 mL, 34.6 mmol) was added. The reaction was heated at reflux for 4 hours. After cooling in an ice bath, methanol (30 mL) was added and the reaction mixture stirred for 0.25 hours. Sodium borohydride (0.92 g, 24.4 mmol) was added and the ice bath removed. The reaction mixture was stirred at ambient temperature for 3 hours then carefully quenched with 1N hydrochloric acid (69 mL, 69 mmol) and extracted with ethyl acetate twice. The combined organic phase was washed with brine, dried (magnesium sulfate), filtered and evaporated to leave an oil. The oil was purified by Horizon Chromatography (silica column) eluting with 0-20% 2M ammonia in methanol-methylene chloride to give the a 3:1 mixture of isomers (3.89 g) ¹H NMR (500 MHz, DMSO): δ 3.22 (0.25H, q, J 6.6), 3.10 (0.75H, q, J 6.6), 2.55 (2H, d, J 7.6), 2.43 (2H, t, J 7.2), 2.40-2.34 (0.75H, m), 2.31-2.23 (0.25H, m), 2.13-2.03 (0.5H, m), 1.98-1.88 (1.5H, m), 1.69-1.61 (2H, m), 1.55-1.43 (3H, m), 1.34-1.18 (1H, m), 1.06 (0.75H, d, J 6.6), 0.96 (2.25H, d, J 6.6), 0.92 (3H, t, J 7.3), 0.81-0.65 (1H, m), 0.50-0.44 (2H, m), 0.13-0.05 (2H, m).

### N-[1-(1-Cyclopropylmethyl-3-propylsulfanylmethyl-cyclobutyl)-ethyl]-2-methoxy-4-methyl-6-trifluoromethyl-nicotinamide

2-Methoxy-4-methyl-6-trifluoromethyl-nicotinic acid (289 mg, 1.23 mmol) in thionyl chloride (3 mL) was heated at reflux for 1 hour. The thionyl chloride was evaporated and the residue azeotroped with toluene (3×) then the 2-methoxy-4-methyl-6-trifluoromethyl-nicotinoyl chloride was dried under vacuum. The nicotinoyl chloride was dissolved in methylene chloride (4 mL) and added to a stirred solution of 1-(1-cyclopropylmethyl-3-propylsulfanylmethyl-cyclobutyl)-ethylamine (100 mg, 0.41 mmol) and triethylamine (0.29 mL, 2.1 mmol) in methylene chloride (5 mL). The reaction mixture was stirred at ambient temperature for 12 hours then the volatile components evaporated. The residue was partitioned between ethyl acetate and water. The organic phase was separated and the aqueous phase was re-extracted with ethyl acetate. The combined organic phase was washed with saturated sodium bicarbonate solution, brine, dried (magnesium sulfate), filtered and evaporated to leave an oil. The oil was purified using the Horizon Chromatography System (silica column) eluting with 0-60% ethyl acetate-hexane to give a oil (138 mg, 73%) as a 3:1 mixture of isomers ¹H NMR (500 MHz, CDCl3): δ 7.14 (1H, s), 6.18-6.12 (1H, m, N—H), 4.58-4.35 (1H, m), 3.98 (2.25H, s), 3.97 (0.75H, s), 2.63-2.57 (2H, m), 2.50-2.42 (6H, m), 2.24-1.98 (2H, m), 1.75-1.65 (2H, m), 1.60-1.53 (3H, m), 1.49-1.31 (1H, m), 1.20 (0.75H, d, J 6.3), 1.11 (2.25H, d, J 6.7), 1.00-0.94 (3H, m), 0.81-0.69 (1H, m), 0.58-0.40 (2H, m), 0.19-0.09 (2H, m).

### N-{1-[1-Cyclopropylmethyl-3-(propane-1-sulfonylmethyl)-cyclobutyl]-ethyl}-2-methoxy-4-methyl-6-trifluoromethyl-nicotinamide (compound 13)

Oxone (555 mg, 0.90 mmol) was added to a solution of N-[1-(1-cyclopropylmethyl-3-propylsulfanylmethyl-cyclobutyl)-ethyl]-2-methoxy-4-methyl-6-trifluoromethyl-nicotinamide (138 mg, 0.30 mmol) in acetone (3 mL) and water (1 mL). The reaction mixture was heated at reflux for 1.5 hours. After cooling to ambient temperature, the reaction mixture was neutralized with 2N sodium carbonate solution and extracted with diethyl ether twice. The combined organic phase was washed with water, brine, dried (magnesium sulfate), filtered and evaporated to leave an oil. The oil was purified using the Horizon Chromatography System (silica column) eluting with 5-60% ethyl acetate-hexane to give a oil (100 mg, 68%) as a 3:1 mixture of isomers ¹H NMR (500 MHz, CDCl3): δ 7.15 (1H, s), 6.11 (1H, d, J 9.3, N—H), 4.64-4.58 (0.25H, m), 4.45-4.39 (0.75H, m), 4.00-3.97 (3H, m), 3.10-3.08 (2H, m), 2.94-2.86 (3H, m), 2.45-2.42 (3H, m), 2.30-2.18 (2H, m), 1.97-1.83 (4H, m), 1.69-1.62 (0.75H, m), 1.49-1.31 (1.25H, m), 1.23-1.19 (0.75H, m), 1.12-1.06 (5.25H, m), 0.81-0.69 (1H, m), 0.61-0.53 (1H, m), 0.49-0.45 (1H, m), 0.20-0.10 (2H, m); MS (m/e)=491.

The following compounds were prepared by the methods outlined in example 13 using the appropriate acid chloride.

| Structure | MS data |
|---|---|
| 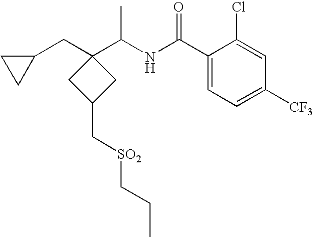 | 480 |
| 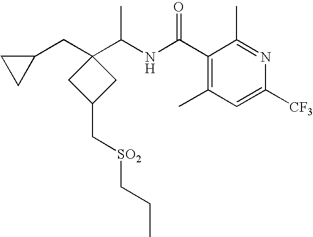 | 475 |

### Example 14

### 2,4-Dichloro-N-[1-piperidin-1-yl-3-(propane-1-sulfonylmethyl)-cyclobutylmethyl]-benzamide (Compound 14)

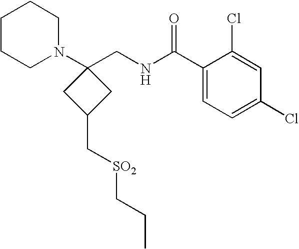

### 3-[(Benzyloxy)methyl]-2,2-dichlorocyclobutanone

To a vigorously stirred suspension of allyl benzyl ether (10.4 mL, 67.5 mmol) and zinc-copper couple (17.6 g, 270 mmol) in anhydrous ether (150 mL) was added a solution of trichloroacetyl chloride (15.1 mL, 135 mmol) in 1,2-dimethoxyethane (14.0 mL, 135 mmol) and ether (100 mL) over a period of 0.5 hours. The mixture was then heated at reflux for 12 hours. The solid was collected by filtration, washing with ether twice. The filtrate was evaporated. The residue was taken up in hexane (300 mL) and filtered (to remove zinc salts). The filtrate was washed with saturated NaHCO₃ solution twice, brine, dried (magnesium sulfate), filtered and evaporated to give an orange oil (15 g, 57.9 mmol) that was used in the next step without further purification.

### 3-[(Benzyloxy)methyl]cyclobutanone

3-[(Benzyloxy)methyl]-2,2-dichlorocyclobutanone (15 g, 57.9 mmol) was dissolved in methanol (150 mL, saturated with NH₄Cl). Zinc (7.57 g, 116 mmol) was added portionwise over 0.5 hours and the mixture stirred at ambient temperature for 3 hours. The solid was collected by filtration washing with ether and the filtrate evaporated. The residue was partitioned between water (100 mL) and ether (250 mL). The ether layer was washed with brine, dried (magnesium sulfate), filtered and evaporated to give a yellow liquid. The crude liquid was chromatographed (silica column) eluting with 10% ethyl acetate-hexane to give a colourless liquid (4.0 g) that was used in the next step without further purification.

### 3-[(Benzyloxy)methyl]-1-piperidin-1-ylcyclobutanecarbonitrile

Sodium cyanide (0.49 g, 9.99 mmol) was added to a stirred solution of piperidine (0.99 ml, 9.99 mmol) in 1N HCl (10 mL) at 0° C. A solution of 3-[(benzyloxy)methyl]cyclobutanone (1.9 g, 9.99 mmol) in methanol (20 mL) was added dropwise over 0.5 hours. On completion of the addition, the mixture was allowed to warm to ambient temperature and stirred for 18 hours. The reaction mixture was poured into ethyl acetate (50 mL) and the mixture carefully neutralised with sodium carbonate solution (2N). The aqueous phase was extracted with ethyl acetate (50 mL) and the combined organics, washed with brine (50 mL), dried (magnesium sulfate), filtered and evaporated to give a colourless liquid (2.8 g) that was used in the next step without further purification.

### 1-{3-[(Benzyloxy)methyl]-1-piperidin-1-ylcyclobutyl}methanamine

To a stirred suspension of lithium aluminium hydride (1.0M solution in ether) (20 mL, 20 mmol) at −78° C. was added a solution of 3-[(benzyloxy)methyl]-1-piperidin-1-ylcyclobutanecarbonitrile (2.8 g, 9.85 mmol) in ether (20 mL) over 20 minutes and the mixture stirred at −78° C. for 1 hour, then allowed to warm to ambient temperature and stirred for 3 hours. The resultant mixture was cooled in an ice bath and water (1 ml) followed by 15% sodium hydroxide solution (2 mL) and finally water (1.0 mL) added. The resultant white granular precipitate was filtered and the solid washed twice with diethyl ether. The filtrated was evaporated to give a colourless oil (2.8 g); MS (m/e)=289.

### N-(3-Benzyloxymethyl-1-piperidin-1-yl-cyclobutylmethyl)-2,4-dichloro-benzamide

To a solution of 1-{3-[(benzyloxy)methyl]-1-piperidin-1-ylcyclobutyl}methanamine (1.5 g, 5.20 mmol) and N-ethyldiisopropylamine (1.08 mL, 6.24 mmol) at 0° C. in methylene chloride (10 mL) was added 2,4-dichlorobenzoyl chloride (0.8 mL, 5.72 mmol) dropwise and the solution allowed to warm to ambient temperature with stirring for 3 hours. Water (4 mL) was added and the reaction stirred for 10 minutes and then passed through a PTFE frit. The organic phase was collected and evaporated to give a pale orange oil (2.2 g); MS (m/e)=361:363 (3:2).

### 2,4-Dichloro-N-(3-hydroxymethyl-1-piperidin-1-yl-cyclobutylmethyl)-benzamide

To a solution of N-(3-benzyloxymethyl-1-piperidin-1-yl-cyclobutylmethyl)-2,4-dichloro-benzamide (2.2 g, 4.77 mmol) in methylene chloride (10 mL) at 0° C. was added trimethylsilyl iodide (3.39 mL, 23.8 mmol) and the solution allowed to warm to ambient temperature with stirring over 1 hour then heated at 60° C. for 16 hours. The reaction mixture was partitioned between ethyl acetate (60 mL) and saturated sodium bicarbonate solution (50 mL). The organic phase was separated and washed with successive portions of saturated sodium bicarbonate solution until neutral. The organic phase was then dried (magnesium sulfate), filtered and evaporated to give an oil. The oil was chromatographed (alumina column) eluting with methylene chloride then 5% methanol-methylene chloride to give a pale orange oil (1.5 g); MS (m/e)=371:373 (3:2).

### Thioacetic acid 3-[(2,4-dichloro-benzoylamino)-methyl]-3-piperidin-1-yl-cyclobutylmethyl ester

Diisopropyl azodicarboxylate (0.53 mL, 2.69 mmol) was added to a solution of triphenylphosphine (0.71 g, 2.69 mmol) in THF (20 mL) at 0° C. and the mixture stirred for 1 hour. A solution of 2,4-dichloro-N-(3-hydroxymethyl-1-piperidin-1-yl-cyclobutylmethyl)-benzamide (0.5 g, 1.35 mmol) and thiolacetic acid (0.19 mL, 2.69 mmol) in THF (10 mL) was added dropwise and the mixture stirred for 1 hour at 0° C. and then allowed to warm to ambient temperature. The reaction was partitioned between ethyl acetate (50 mL) and water (25 mL) and the organic phase separated, dried (magnesium sulfate), filtered and evaporated to give a yellow oil. The crude product was chromatographed (alumina) eluting with methylene chloride then 5% methanol-methylene chloride to give the product as a pale yellow oil (0.480 g); MS (m/e)=429:431 (3:2).

### 2,4-Dichloro-N-(1-piperidin-1-yl-3-propylsulfanylmethyl-cyclobutylmethyl)-benzamide

To a solution of thioacetic acid 3-[(2,4-dichloro-benzoylamino)-methyl]-3-piperidin-1-yl-cyclobutylmethyl ester (0.48 g, 1.12 mmol) in isopropyl alcohol (10 mL) and 1-bromopropane (0.20 mL, 2.24 mmol) was added a solution of lithium hydroxide (0.11 g, 4.47 mmol) in water (2.0 mL) and the mixture stirred at ambient temperature for 1 hour. The reaction mixture was partitioned between methylene chloride (30 mL) and water (15 mL) and the organic phase separated, dried (magnesium sulfate), filtered and evaporated to give a colourless oil (170 mg); MS (m/e)=429:431 (3:2).

### 2,4-Dichloro-N-[1-piperidin-1-yl-3-(propane-1-sulfonylmethyl)-cyclobutylmethyl]-benzamide

To a solution of 2,4-dichloro-N-(1-piperidin-1-yl-3-propylsulfanylmethyl-cyclobutylmethyl)-benzamide (0.17 g, 0.40 mmol) in acetone (6 mL) was added trifluoroacetic acid (0.030 mL, 0.40 mmol) followed by a solution of oxone (0.73 g, 1.19 mmol) in water (2 mL) and the solution heated at reflux for 2 hours. The reaction was poured onto an SCX cartridge and eluted with methanol followed by 2M ammonia in methanol to give an oil which was purified by preparative TLC (silica) eluting with 5% methanol-methylene chloride to give the trans isomer as a white solid ¹H NMR δ (ppm) 500 MHz (CDCl₃): 7.67 (1H, d, J 8.3), 7.43 (1H, d, J 1.9), 7.33 (1H, dd, J 1.9, 8.3), 6.82 (1H, s, N—H), 3.59 (2H, s), 3.15 (2H, d, J 7.4), 2.91-2.85 (3H, m), 2.61 (2H, m), 2.57 (4H, m), 1.91-1.83 (2H, m), 1.78 (2H, m), 1.58 (4H, m), 1.46 (2H, m), 1.08 (3H, t, J 7.4); MS (m/e)=461:463 (3:2); from NOE experiments methylsulfone and piperidine are trans; and the cis isomer as a white solid ¹H NMR δ (ppm) 400 MHz (CDCl₃): 7.68 (1H, d, J 8.4), 7.43 (1H, d, J 2.0), 7.33 (1H, dd, J 2.0, 8.3), 6.92 (1H, s, N—H), 3.67 (2H, d, J 4.4), 3.08 (2H, m), 2.92-2.88 (2H, m), 2.66 (1H, m), 2.45 (4H, m), 2.10 (4H, m), 1.92-1.82 (2H, m), 1.54 (4H, m), 1.43 (2H, m), 1.09 (3H, t, J 7.4); MS (m/e)=461:463 (3:2); from NOE experiments methylsulfone and piperidine are cis.

1-10. (canceled)

11. A compound of the formula I: 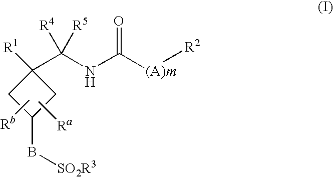
wherein:
R¹ is —(CH₂)ₙ—R^{1a}, wherein n is independently 0-6, and R^{1a} is selected from the group consisting of:
(1) C₁₋₆alkyl or C₁₋₆alkenyl, which is unsubstituted or substituted with 1-6 halogen, hydroxyl or —NR¹⁰R¹¹,
(2) phenyl substituted with R^{2a}, R^{2b} and R^{2c},
(3) heterocycle substituted with R^{2a}, R^{2b} and R^{2c},
(4) C₃₋₆cycloalkyl, which is unsubstituted or substituted with C₁₋₆alkyl, 1-6 halogen, hydroxy or —NR¹⁰R¹¹,
(5) —O—C₁₋₆alkyl, which is unsubstituted or substituted with 1-6 halogen, hydroxy or —NR¹⁰R¹¹,
(6) —CO₂R⁹,
wherein R⁹ is independently selected from:
(a) hydrogen,
(b) —C₁₋₆alkyl, which is unsubstituted or substituted with 1-6 fluoro,
(c) benzyl, and
(d) phenyl,
(7) —NR¹⁰R¹¹,
wherein R¹⁰ and R¹¹ are independently selected from:
(a) hydrogen,
(b) —C₁₋₆alkyl, which is unsubstituted or substituted with hydroxy, 1-6 fluoro or —NR¹²R¹³, where R¹² and R¹³ are independently selected from hydrogen and —C₁₋₆alkyl,
(c) —C₃₋₆cycloalkyl, which is unsubstituted or substituted with hydroxy, 1-6 fluoro or —NR¹²R¹³,
(d) benzyl,
(e) phenyl, and
(8) —CONR¹⁰R¹¹;
R² is selected from the group consisting of:
(1) phenyl, which is substituted with R^{2a}, R^{2b} and R^{2c},
(2) heterocycle, which is substituted with R^{2a}, R^{2b} and R^{2c},
(3) C₁₋₈alkyl, which is unsubstituted or substituted with 1-6 halogen, hydroxy, —NR¹⁰R¹¹, phenyl or heterocycle, where the phenyl or heterocycle is substituted with R^{2a}, R^{2b} and R^{2c},
(4) C₃₋₆cycloalkyl, which is unsubstituted or substituted with 1-6 halogen, hydroxy or —NR¹⁰R¹¹, and
(5) —C₁₋₆alkyl-(C₃₋₆cycloalkyl), which is unsubstituted or substituted with 1-6 halogen, hydroxy or —NR¹⁰R¹¹;
R^{2a}, R^{2b} and R^{2c} are independently selected from the group consisting of:
(1) hydrogen,
(2) halogen,
(3) —C₁₋₆alkyl, which is unsubstituted or substituted with:
(a) 1-6 halogen,
(b) phenyl,
(c) C₃₋₆cycloalkyl, or
(d) —NR¹⁰R¹¹,
(4) —O—C₁₋₆alkyl, which is unsubstituted or substituted with 1-6 halogen,
(5) hydroxy,
(6) —SCF₃,
(7) —SCHF₂,
(8) —SCH₃,
(9) —CO₂R⁹,
(10) —CN,
(11) —SO₂R⁹,
(12) —SO₂—NR¹⁰R¹¹;
(13) —NR¹⁰R¹¹,
(14) —CONR¹⁰R¹¹, and
(15) —NO₂;
R³ is selected from the group consisting of:
(1) C₁₋₆alkyl, which is unsubstituted or substituted with 1-6 halogen, hydroxyl, —NR¹⁰R¹¹, or heterocycle, which is substituted with R^{2a}, R^{2b} and R^{2c},
(2) C₃₋₆cycloalkyl, which is unsubstituted or substituted with 1-6 halogen, hydroxyl or —NR¹⁰R¹¹;
(3) —C₁₋₆alkyl-(C₃₋₆cycloalkyl), which is unsubstituted or substituted with 1-6 halogen, hydroxy or —NR¹⁰R¹¹,
(4) —NR¹⁰R¹¹, and
(5) heterocycle, which is substituted with R^{2a}, R^{2b} and R^{2c};
R⁴ and R⁵ are each independently selected from the group consisting of:
(1) hydrogen, and
(2) C₁₋₆alkyl, which is unsubstituted or substituted with halogen or hydroxyl;
A is selected from the group consisting of:
(1) —O—, and
(2) —NR¹⁰—;
m is zero or one;
B is selected from the group consisting of
(1) —CR⁶R⁷—, and
(2) —NR⁸
wherein R⁶, R⁷ and R⁸ are each independently selected from hydrogen and C₁₋₆alkyl; R^{a} and R^{b} are each independently selected from hydrogen and C₁₋₄alkyl when B is NR⁸ and are each independently selected from hydrogen, fluorine, chlorine and C₁₋₄alkyl when B is CR⁶R⁷; or a pharmaceutically acceptable salt thereof.

12. The compound of claim 11 of the formula Ia: 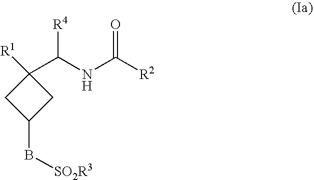
or a pharmaceutically acceptable salt thereof.

13. The compound of claim 11 of the formula Ib: 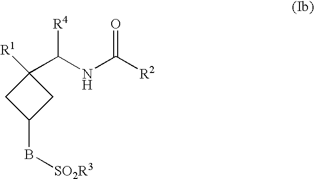
wherein:
R² is phenyl or unsaturated heterocycle substituted with R^{2a}, R^{2b} and R^{2c}, B is CHR⁷ or NR⁸ and R^{2a}, R^{2b} and R^{2c} are selected from hydrogen, fluoro, chloro, bromo, CH₃, OCH₃, CF₃, OCF₃ and NH₂;
or a pharmaceutically acceptable salt thereof.

14. The compound of claim 11 of the formula Ic: 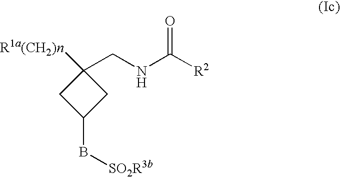
wherein:
R² is phenyl or unsaturated heterocycle substituted with R^{2a}, R^{2b} and R^{2c}, and R^{3b} is a C₁₋₄ alkyl group optionally substituted by a C₃₋₆cycloalkyl group;
or a pharmaceutically acceptable salt thereof.

15. The compound of claim 11 of the formula Id: 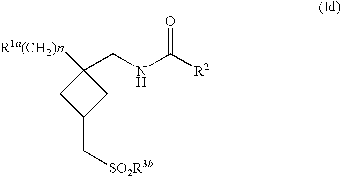
wherein:
R² is phenyl or unsaturated heterocycle substituted with R^{2a}, R^{2b} and R^{2c};
or a pharmaceutically acceptable salt thereof.

16. The compound of claim 11 wherein R^{1a} is C₃₋₆cycloalkyl, which is unsubstituted or substituted with C₁₋₆alkyl, 1-6 halogen, hydroxy or —NR¹⁰R¹¹.

17. The compound of claim 11 wherein R^{2a}, R^{2b}, R^{2c} are selected from the group consisting of hydrogen, OCH₃, CH₃, CF₃ or halogen,

18. The compound of claim 11 wherein R^{2a}, R^{2b}, R^{2c} are chlorine or fluorine.

19. The compound of claim 11 wherein R^{3b} is a C₁₋₄ alkyl group optionally substituted by a cyclopropyl group.

20. A compound which is selected from the group consisting of: 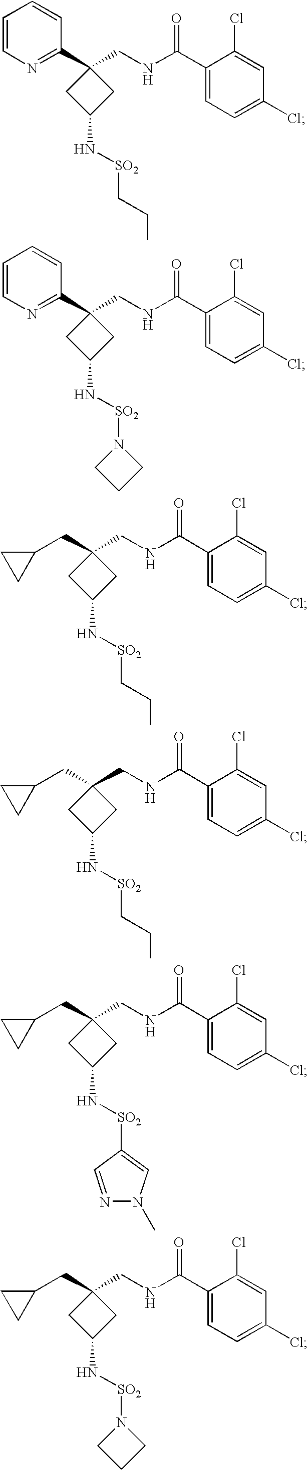 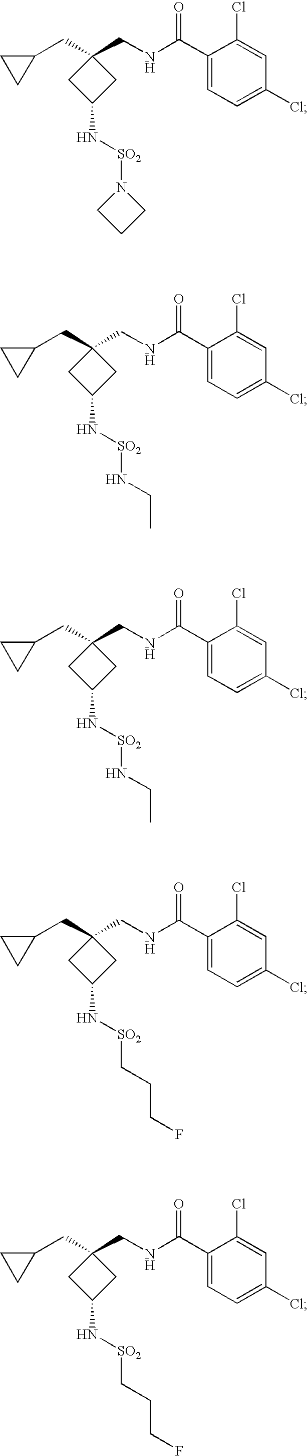 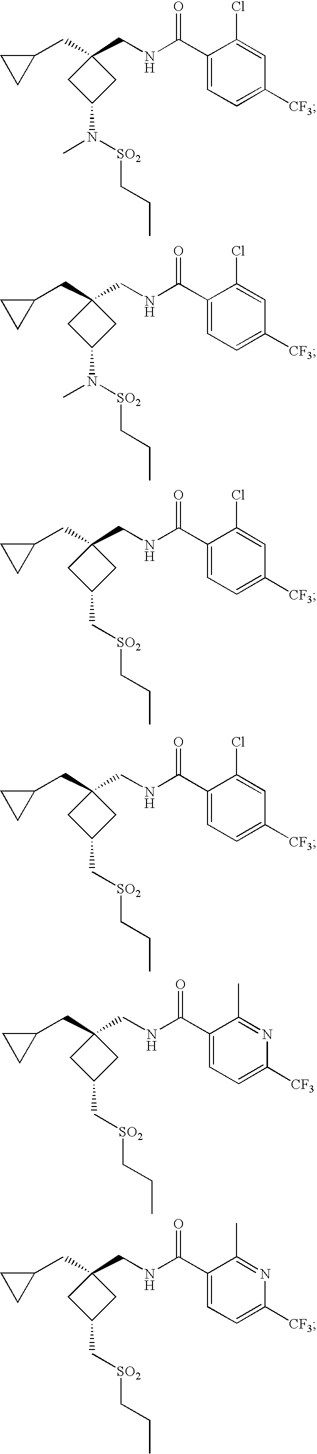 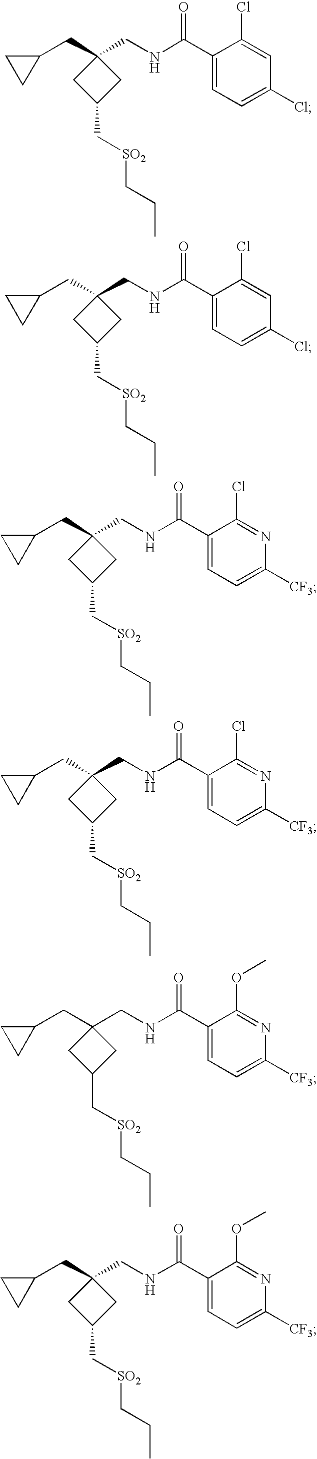 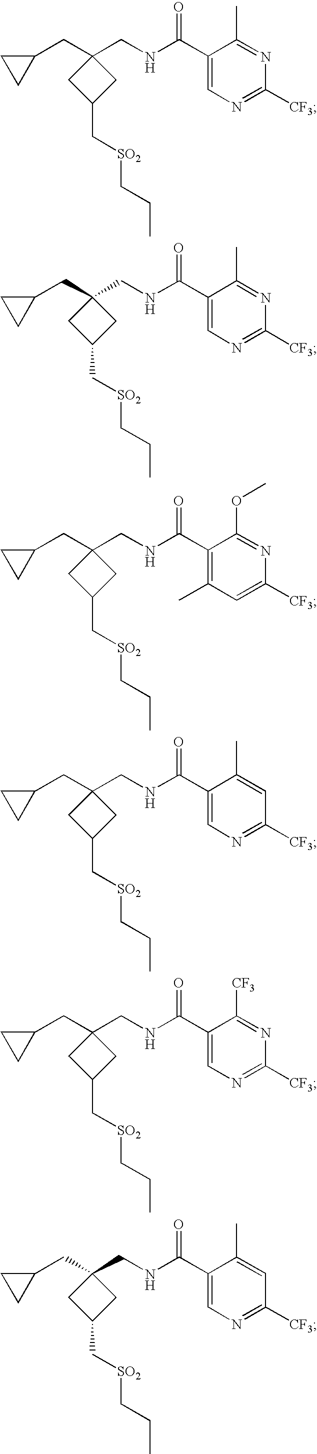 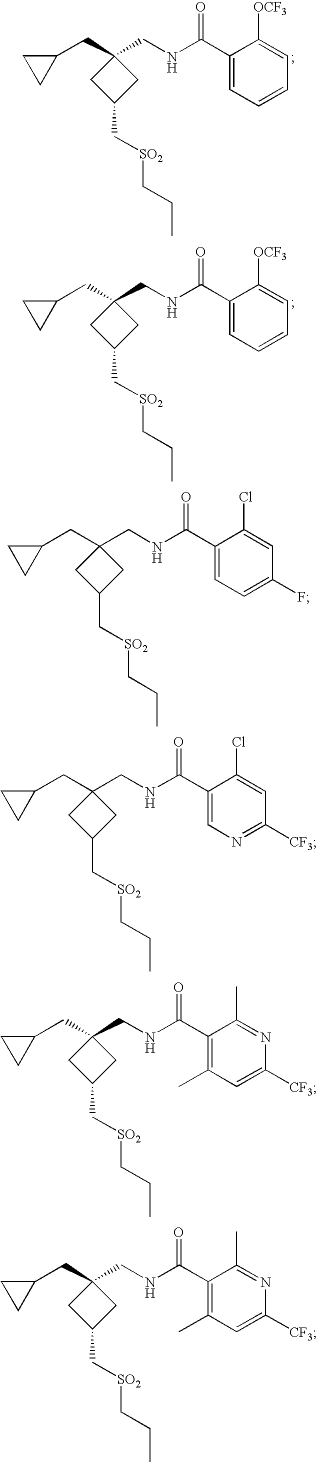 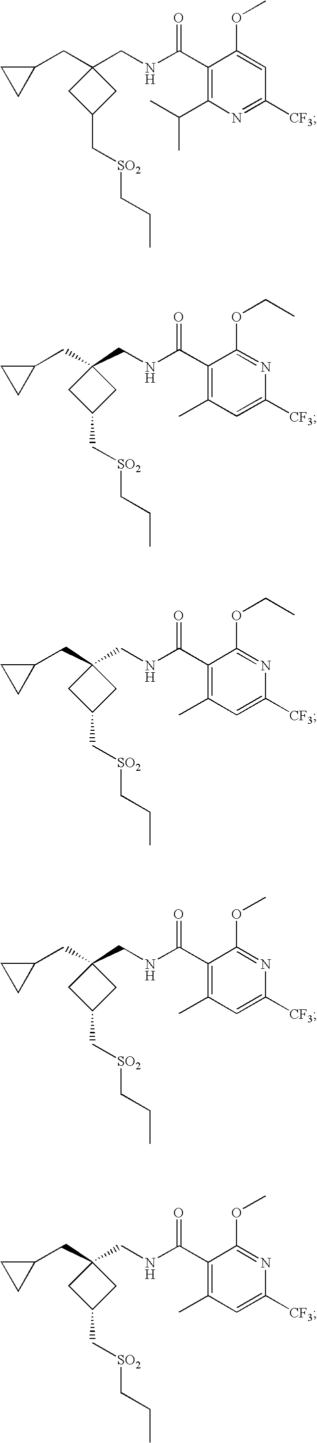 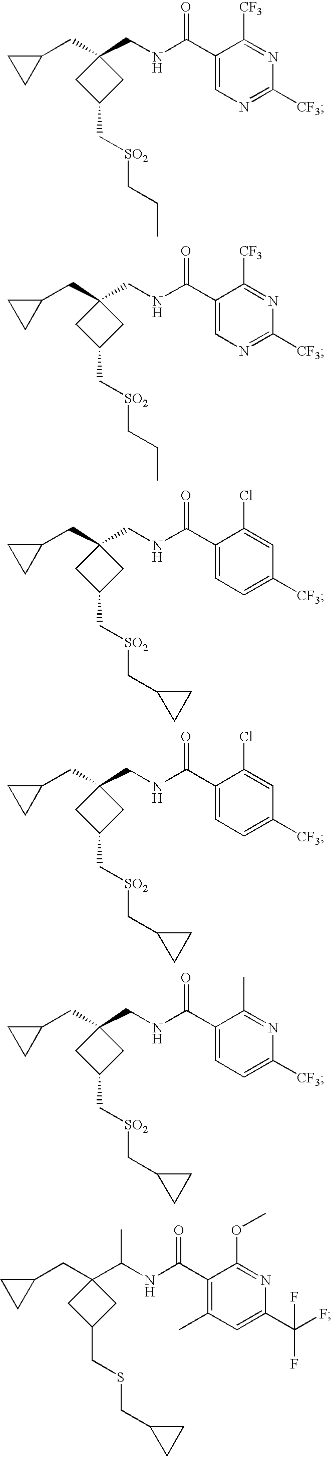 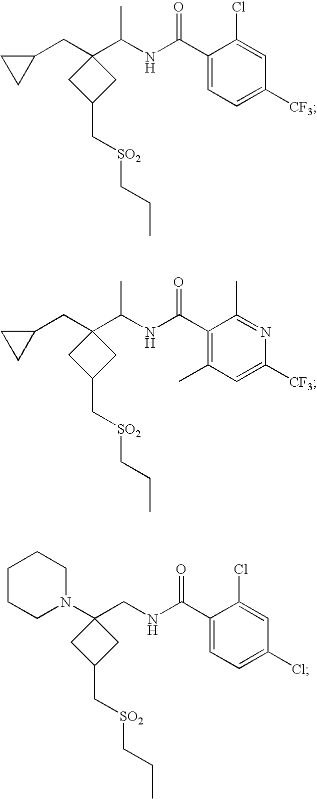
or a pharmaceutically acceptable salt thereof,

21. A pharmaceutical composition comprising the compound of claim 11, or a pharmaceutically acceptable salt thereof, and a pharmaceutically acceptable excipient.

22. A method for treating a neurological or a psychiatric disorder associated with glycinergic or glutamatergic neurotransmission dysfunction in a mammalian patient in need thereof which comprises administering to the patient a therapeutically effective amount of a compound of claim 11 or a pharmaceutically acceptable salt thereof.

23. A method for treating schizophrenia in a mammalian patient in need thereof which comprises administering to the patient a therapeutically effective amount of a compound of claim 11 or a pharmaceutically acceptable salt thereof.
